(19) United States
(12) Patent Application Publication
(10) Pub. No.: US 2014/0099700 A1
(43) Pub. Date: Apr. 10, 2014

(54) APPARATUS AND SYSTEM FOR BIOREACTOR CULTIVATING MICROORGANISMS AND REDUCING CARBON DIOXIDE

(71) Applicant: ENERGAIA, INC., Atlanta, GA (US)

(72) Inventors: Saumil Navnit Shah, Bangkok (TH); Derek Andrew Blitz, Bangkok (TH); Patsakorn Thaveeuchukorn, Bangkok (TH)

(73) Assignee: ENERGAIA, INC., Atlanta, GA (US)

(21) Appl. No.: 13/644,582

(22) Filed: Oct. 4, 2012

(51) Int. Cl. C12M 1/04; C12N 1/02; C12M 1/12; C12S 5/00; C12M 1/00; C12M 1/42

(52) U.S. Cl. 435/261; 435/289.1; 435/292.1; 435/297.1; 435/266

(57) ABSTRACT

A bioreactor includes a support structure and a translucent liner. A containing system of bioreactors includes a plurality of bioreactors. A contained bioreactor system includes a plurality of containing systems of interconnected bioreactors, an industrial blower, an air filter, a microbial filter, a gas delivery system, and a harvesting station. A method of reducing carbon dioxide emissions includes delivering carbon dioxide to a bioreactor where the bioreactor includes a support structure and a liner where the bioreactor includes media containing microbes. A method of harvesting microorganisms includes emptying the solution and removing a sieve from the harvesting unit.

### FIELD

The present disclosure generally relates to a bioreactor for cultivating microorganisms and reducing carbon dioxide from various systems as well as apparatus, systems, and methods concerning bioreactors.

### BACKGROUND

Industry worldwide is causing an increase in carbon dioxide emissions. The increase in carbon dioxide emissions worldwide is believed to contribute to global warming and also alter the climate throughout the world. In some cases, carbon dioxide emissions can pose a cost to businesses in the form of tax penalties, environmental regulations, and countermeasures.

### SUMMARY

A bioreactor includes a support structure and a liner where the liner is fitted within the support structure.

A containing system of bioreactors includes a plurality of interconnected bioreactors.

A containing system includes a plurality of containing systems of interconnected bioreactors, an industrial blower, an air filter, a microbial filter, a gas delivery system, and a harvesting unit.

A method of reducing carbon dioxide emissions includes delivering gaseous carbon dioxide to a bioreactor where the bioreactor includes a support structure and a transparent liner and where the bioreactor includes media containing microbes that consume gaseous carbon dioxide as a carbon source; and growing microbes in the bioreactor.

A method of harvesting microorganisms from a containing system includes emptying the solution from the solution outlet of a harvesting unit of a containing system where the containing system is made of a plurality of bioreactors where each bioreactor is made of a support structure and a transparent liner, and where the harvesting unit is closed off from all the other bioreactors in the containing system, and removing a sieve from the harvesting unit where the sieve contains microbes.

### DESCRIPTION OF THE FIGURES

The features and components of the following figures are illustrated to emphasize the general principles of the present disclosure and are not necessarily drawn to scale. Corresponding features and components throughout the figures may be designated by matching reference characters for the sake of consistency and clarity.

FIG. 1 is a cross sectional view of a foundation for placement of a bioreactor in accord with one embodiment of the current disclosure.

FIG. 2 is a top view of the foundation of FIG. 1.

FIG. 3 is a schematic view of a rooftop embodiment of a bioreactor system in accord with one embodiment of the current disclosure where appropriate.

FIG. 4 is a side view of one bioreactor and one embodiment of its foundation.

FIG. 5 is a cross-sectional view of the bioreactor and foundation of FIG. 4.

FIG. 6 is a perspective view of a support structure in accord with one embodiment of the current disclosure.

FIG. 7 is a detail view of detail 7 in FIG. 6.

FIG. 8 is the side view of the support structure of FIG. 6.

FIG. 9 is a detail view of detail 9 in FIG. 8.

FIG. 10 is a side view of an inflated liner in accord with one embodiment of the current disclosure.

FIG. 11 is a perspective view of the liner of FIG. 10 inflated and opened to accept the shape of the support of FIG. 6.

FIG. 12 is the perspective view of the liner of FIG. 10.

FIG. 13 is the side view the liner of FIG. 10.

FIG. 14 is the front view of the liner of FIG. 10.

FIG. 15 is the perspective view of the inflated liner of FIG. 15.

FIG. 16 is the bottom view of the liner of FIG. 10.

FIG. 17 is the side view of the inflated liner of FIG. 11.

FIG. 18 is the side view of the support of FIG. 8 with the liner of FIG. 11.

FIG. 19 is the perspective view of the wire mesh support of FIG. with the liner of FIG. 11.

FIG. 20 is a side view of an aeration pump in accord with one embodiment of the current disclosure.

FIG. 21 is the front view of the up-pipe of the aeration pump of FIG. 20.

FIG. 22 is the side view of the aeration pump of FIG. 20.

FIG. 23 is a detail cross-sectional view of detail 23 of FIG. 22.

FIG. 24 is a perspective view of the aeration pump of FIG. 20.

FIG. 25 is the side view of the aeration pump of FIG. 20.

FIG. 26 is a detail cross-sectional view of detail 26 of FIG. 25.

FIG. 27 is a side cross-sectional view of a conduit fitting connector exploded into its individual parts.

FIG. 28 is a side view of the conduit fitting connector of FIG. 27.

FIG. 29 is a cross-sectional view of the conduit fitting connector of FIG. 27.

FIG. 30 is a rear facing, perspective view of the conduit fitting connector of FIG. 27.

FIG. 31 is a front facing, perspective view of the conduit fitting connector of FIG. 27.

FIG. 32 is a perspective view of the bioreactor cover in accord with one embodiment of the current disclosure.

FIG. 33 is a top view of the bioreactor cover of FIG. 32.

FIG. 34 is a side view of the bioreactor cover of FIG. 32.

FIG. 35 is a top view of a bioreactor cover of FIG. 32.

FIG. 36 is a perspective view of a bioreactor cover of FIG. 32.

FIG. 37 is a cross-sectional view of a solution injector in accord with one embodiment of the current disclosure.

FIG. 38 is a front view of the solution injector of FIG. 37.

FIG. 39 is a side view of the solution injector of FIG. 37.

FIG. 40 is a detail cross-sectional view of detail 40 of FIG. 37.

FIG. 41 is a top view of three interconnected bioreactors in accord with one embodiment of the current disclosure.

FIG. 42 is a perspective view of the interior of the bioreactor interconnected in accord with one embodiment of the current disclosure.

FIG. 43 is a perspective view of the interior of the bioreactor of FIG. 42.

FIG. 44 is a top perspective view of the interior of the bioreactor of FIG. 42.

FIG. 45 is a schematic of the gas delivery system in accord with one embodiment of the current disclosure.

FIG. 46 is a schematic for the flue gas heat exchange in accord with one embodiment of the current disclosure.

FIG. 47 is a cross-sectional view of a fixed time harvesting unit in accord with one embodiment of the current disclosure.

FIG. 48 is a cross-sectional view of a continuous harvesting unit in accord with one embodiment of the current disclosure.

FIG. 49 is a cross-sectional view of a harvesting unit in accord with one embodiment of the current disclosure.

FIG. 50 is a side view of the three interconnected bioreactors of FIG. 41

FIG. 51 is a chart comparing Spirulina density from Spirulina cultures given different amounts of NaCHO₃ and carbon dioxide.

FIG. 52 is a chart comparing Spirulina dry weight from Spirulina cultures given different amounts of NaCHO₃ and carbon dioxide.

FIG. 53 is a chart comparing Spirulina density from Spirulina cultures given different amounts of NaCHO₃.

FIG. 54 is a chart comparing Spirulina dry weight from Spirulina cultures given different amounts of NaCHO₃.

### DETAILED DESCRIPTION

The present disclosure will now be described more fully hereinafter with reference to the accompanying drawings, in which some, but not all embodiments of the disclosure are shown. Indeed this disclosure may be embodied in many different forms and should not be construed as limited to the embodiments set forth hereinafter; rather, these embodiments are provided so that this disclosure will satisfy applicable legal requirements. Like numbers refer to like elements throughout.

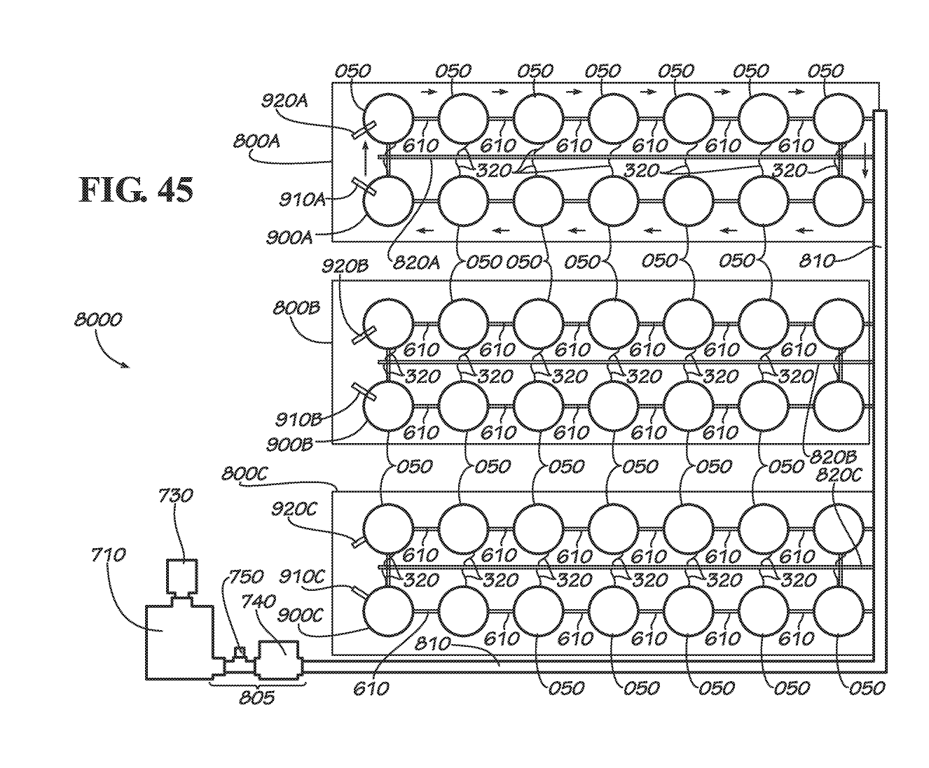

Various apparatus and systems (as depicted in FIG. 45, 50) are described that utilize atmospheric and industrial carbon dioxide to cultivate microorganisms in a bioreactor module 050. The bioreactor module 050 is comprised of a temporary support structure 100 and a liner 250 that is flexible and transparent or translucent. The apparatus may be filled with media that supports the cultivation of microorganisms. Carbon dioxide is fed into the apparatus and feeds the microorganisms. Multiple bioreactor modules 050 may comprise a containing system 800 wherein a large amount of atmospheric carbon dioxide will be cultivated into microorganisms and later harvested.

The present disclosure also describes a containing system 800 that is designed to cultivate microorganisms (such as algae) and consume carbon dioxide both from air and flue gas using the aforementioned bioreactor module 050. In one embodiment, the system includes individual bioreactor modules 050 interconnected by airlift pumps 310 that create gas absorption and fluid/microorganism flow between individual bioreactor modules 050. The interconnected bioreactor modules 050 form a containing system 800 of interconnected bioreactor modules 050 that is used to cultivate and to harvest microorganisms with decreased labor, lower costs, and increased efficiency as compared to some other methods. Multiple containing systems 800 of interconnected individual bioreactor modules 050 can form a larger overall system 8000 that is scalable in size from a small rooftop system, for example, to multiple thousand bioreactor modules 050 on large industrial or agricultural sites, for example.

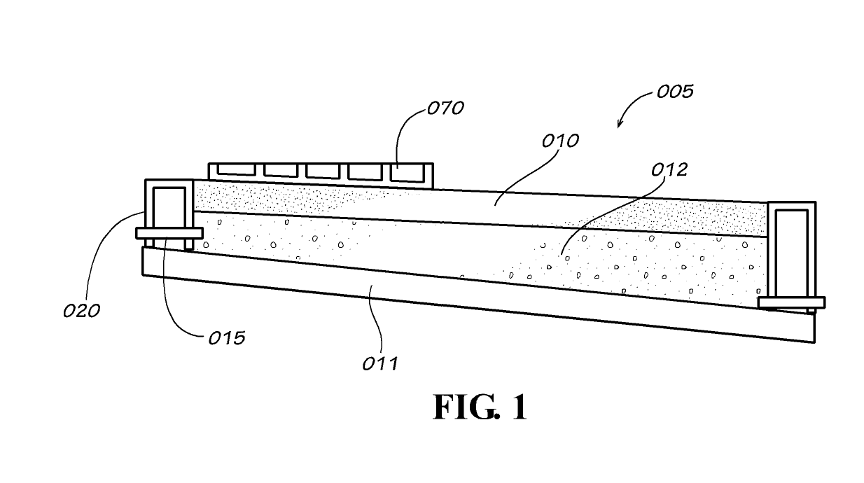

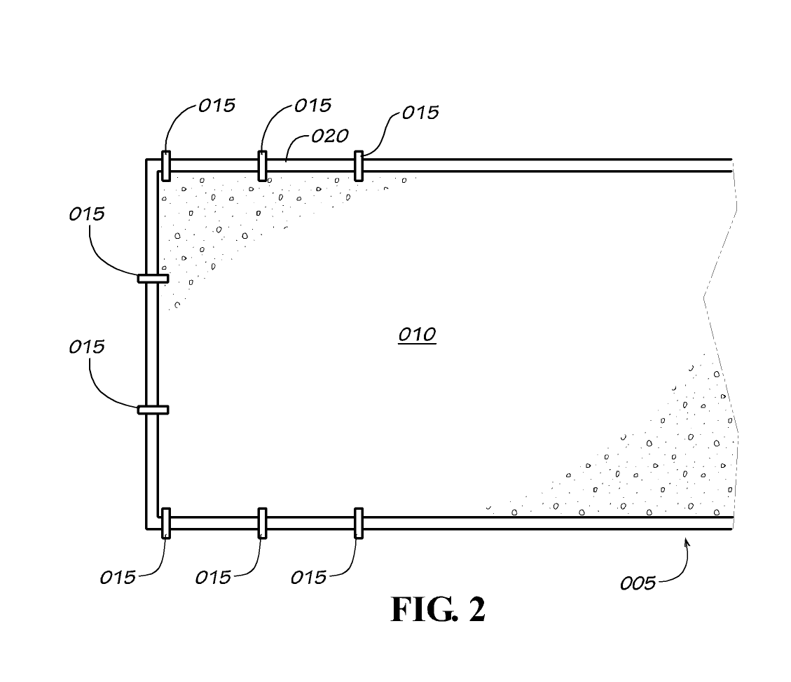

FIG. 1 shows one embodiment of a finished foundation 005. A base 010 is formed whereupon the bioreactor modules 050 will be positioned. The foundation 005 is built up over the existing ground 011. Gravel, earth, and/or concrete 012 is built up over the existing ground 011 and levelled. Weep holes 015 may be used to allow drainage. The foundation may need retaining walls 020 to hold in the added gravel, earth, or concrete 012. Then additional gravel or crushed dust is added to the top finishing the base 010 (as seen in FIG. 2).

The bioreactor module 050 of this disclosure should be situated where the foundation 005 is stable enough to support any load of the system and maintain such support. The bioreactor modules 050 can sit on earth, gravel, concrete, or even the rooftop of a building. The foundation 005 should be able to support the load through inclement weather conditions. Some sites may be prone to greater rain or flooding than other areas and may have the risk that some earth may wash away under a portion of the bioreactor module 050, leaving the bioreactor module 050 with the risk of tipping over. To lower this risk, the site may be prepped for drainage, and the ground may be covered with a thin plastic barrier to prevent muddy foundations.

Sites should be prepared for installation of bioreactor modules 050 and an air delivery system 805. In the current embodiment, each bioreactor module 050 should be on a level surface. In one embodiment, the entire system 805 will be installed on levelled ground. In another embodiment, where the terrain does not permit level installation, the ground may be stepped in such a way that individual bioreactor modules 050 reside at different vertical levels even though each bioreactor module 050 contained within any given containing system 800 is at the same level as the others in its containing system 800. In some embodiments, the bioreactor modules 050 may be placed on metal or plastic pallets 070.

For earthen installations, the site may be bulldozed and levelled. Depending on the earthen material, gravel, earth, and/or concrete 012 may be used to reinforce the base 010 or surface of the foundation 005. The site should be levelled in such a way that rainfall and natural elements will not wash away surface matter causing uneven foundation for the bioreactor module 050. In some cases, the use of retaining walls 020 and fill is necessary while in others site preparation is limited to simple clean up.

FIG. 2 is an aerial view of the foundation. In this embodiment, the perimeter has retaining walls 020 surrounding the perimeter. There are also weep holes 015 that allow drainage.

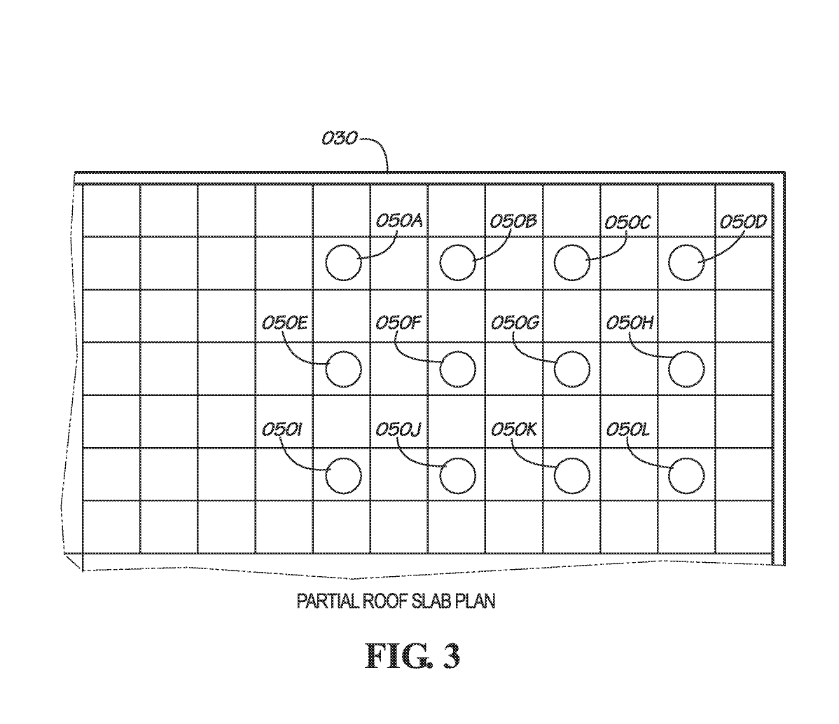

Bioreactor modules 050A-L and containing systems 800 may also be installed on rooftops, as depicted in FIG. 3. In many cases, flat rooftops are designed to support the load of the liquid contained within the system. When rooftops are not designed to withstand the additional load, shorter bioreactor modules 050 may be used, and support structures may be built as platforms that evenly distribute the load over the rooftop. In one embodiment, the bioreactor modules 050 are placed two meters apart on a concrete slab on a rooftop between roof parapets 030.

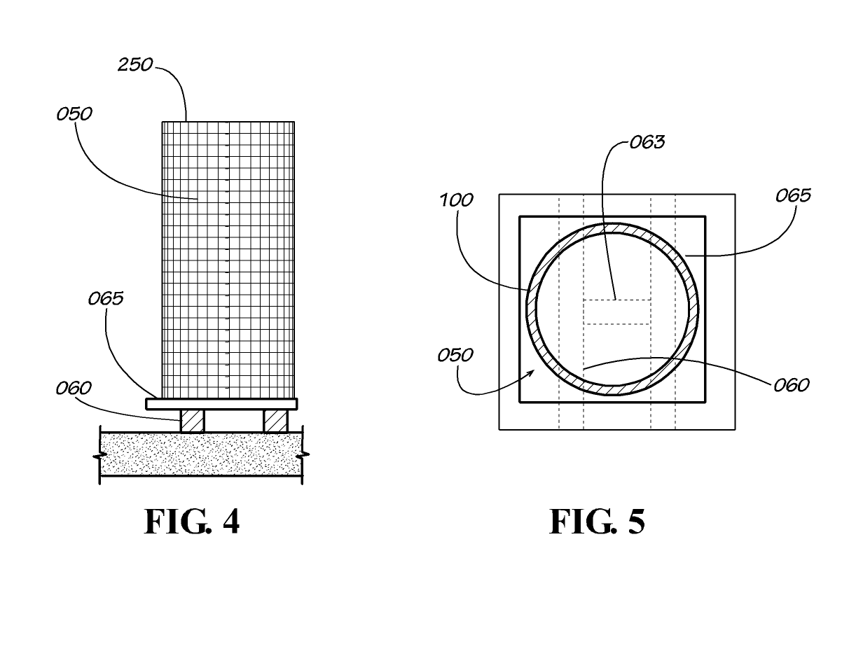

When bioreactor modules 050 are on a rooftop, wood or sheet material may be used to serve as a leveller or foundation. One such example is seen in FIGS. 4 and 5. In one embodiment, a 7.5 cm by 10 cm by 100 cm long wood sleeper 060 with a tie member 063 can act as a foundation for a bioreactor module 050. A one half inch support 065, which may be plywood in some embodiments, may be placed on top of the wood sleeper 060 to provide a level foundation and support for the bioreactor module 050.

In another embodiment, the bioreactor system site may have (1) access to a water source, wherein the water source may be a river, lake, well, or municipal water; (2) access to electricity in order to run a gas delivery system; in some embodiments, photovoltaic cells or other natural electricity sources may be used; (3) the ability to build a storage shelter that protects the air blower and electrical system; other replacement fittings and materials can also be kept in this shelter; and (4) access to flue gas from an industrial source, although such a source of gas is not required for effective functioning of the system. While there are advantages to these elements, some embodiments may include fewer elements than those listed in this paragraph.

One advantage of the bioreactor module 050 and bioreactor system of the present disclosure is that it can be implemented in many locations. Many other bioreactors that cultivate microorganisms are limited in where they can be implemented. Some bioreactors are small ponds that require land, space, a water source, and proper drainage, among other considerations. Many industrial bioreactors require large power and carbon sources. In contrast, the bioreactor module 050 and system of the present disclosure can be scaled to many different needs and circumstances. In another embodiment, the bioreactor module 050 will be near the flue gas of an industrial source. Since the system includes multiple bioreactor modules 050, the size of the system can be flexible with the conditions of the site.

The microorganisms cultivated for many other bioreactor systems are typically cultivated in open pond systems or in very costly closed systems. Both approaches present a problem because each leads to increased costs in providing carbon nutrient sources, electricity for mixing, and contamination, among other challenges. The embodiments of the current disclosure offer a solution to cultivate microorganisms in a semi-closed system that is cost effective to set up and to operate. Further, the systems of the current disclosure are designed to deliver very high microbial growth rates by optimizing mixing, gas absorption, and nutrient delivery. Compared to other systems designed to achieve the same growth rates, the design of the current disclosure is optimized to be much lower cost for both set up and operation, making it commercially viable and scalable.

FIG. 5 shows an embodiment of the structural support 100 of the bioreactor module 050. In one embodiment, the bioreactor module 050 is supported by a moveable support structure 100 made from a semi rigid material that can support the vertical load of the liquid and give the bag its cylindrical shape. In one embodiment, this may be a wire mesh, welded wire mesh (12-16 AWG), or equivalent structure. This structure can be formed into any cross sectional shape. In one embodiment, the support structure 100 is galvanized or PVC coated wire mesh bent into a cylinder.

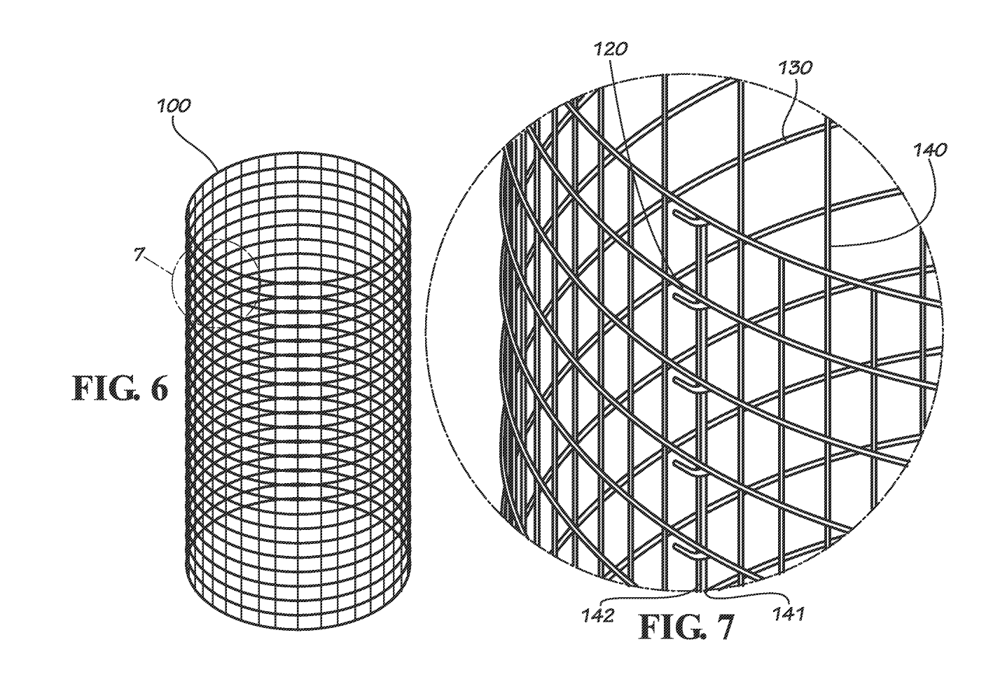

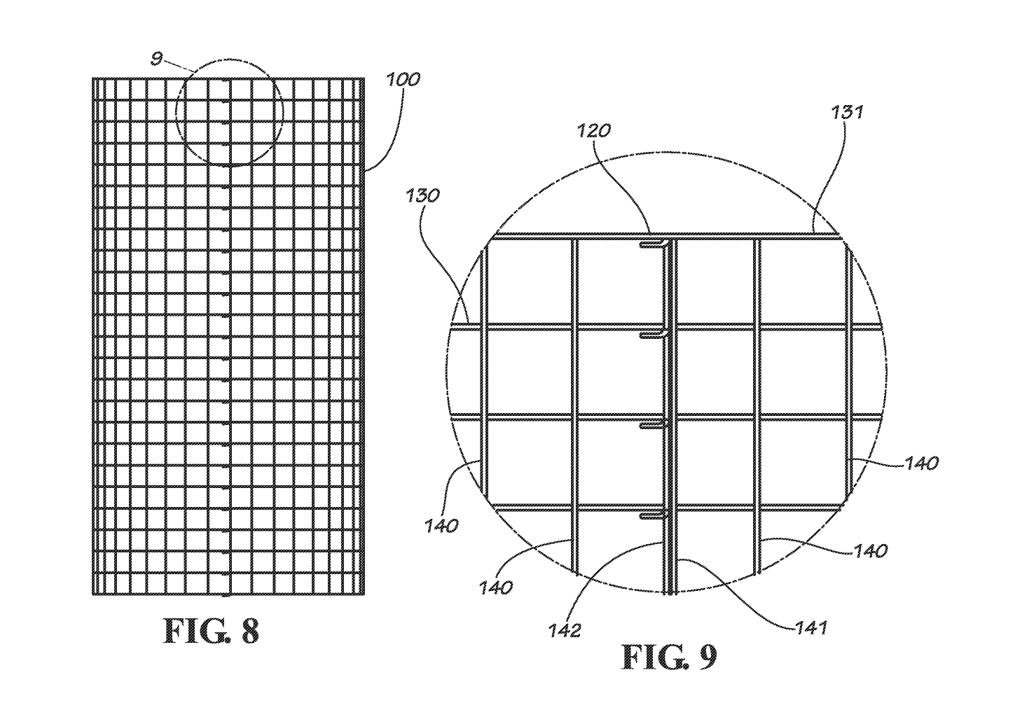

In one embodiment, as depicted in FIG. 6, the support structure 100 is formed from a wire mesh, or flat sheet from a roll. The mesh is cut to a length to match the circumference of the liner 250 plus the width of two squares of wire mesh. FIG. 7 shows an embodiment of the of the support structure 100 as the ends are connected. The horizontal members 130 protruding from one end such that there is only one additional vertical member 141 beyond the liner circumference length. Protruding wires 120 from the horizontal members 130 are used to fasten the mesh into a cylinder by way of wrapping around a second vertical member 142, as depicted in FIG. 7. The support structure 100 may also be fastened together with zip ties or welding. Other methods may be used to create a semi rigid cylinder. FIG. 8 shows a side view of the support structure 100. It is cylindrical and upright.

In this embodiment, the protruding wire 120 is wrapped around the second vertical member 142 on the opposing end of the protruding wires 120 as shown in FIG. 9. Wire is wrapped in such a way that the mesh support structure 100 becomes a semi rigid support structure capable of containing the load of liner 250 and contained solution Excess wire is bent outwards as not to puncture liner 250. The protruding wires 120 are wrapped around vertical members 142 to fasten the bent cylinder to itself. The support structure 100 is wrapped into a cylinder with the horizontal members 130 inside the vertical members 140 and the horizontal members 130 acting as a strap containing the liner 250 and contained liquid. Horizontal members 130 provide shape and structure to the wire mesh support structure 100. The vertical members 140 are contained within, and welded to, the horizontal members 130. The vertical wire members 140 provide vertical support and stability to the wire mesh support 100.

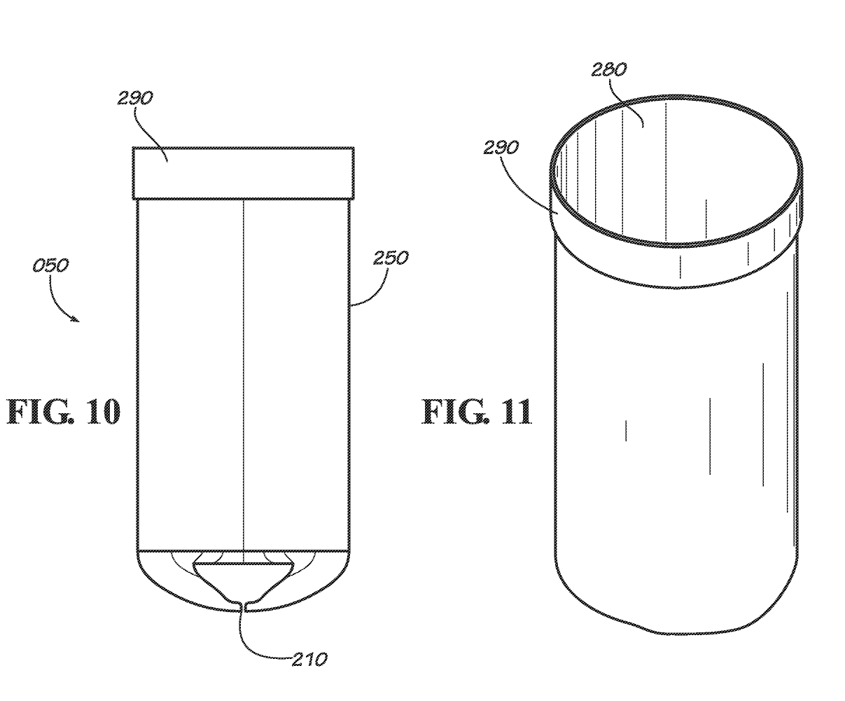

FIG. 10 shows a side view of a transparent liner 250. The bioreactor module 050 includes the liner 250, which may be flexible and transparent or translucent in various embodiments. In one embodiment, the liner 250 is a flexible, transparent plastic bag. FIG. 10 shows that the bottom corners 210 of the liner 250 are thermally sealed. The position may be temporarily fastened with tape. By this method, the liner 250 fills the wire mesh support 100 almost entirely and, when filled with water, assumes the shape of the support structure 100 forming a transparent cylinder. The weight of the water creates pressure on the bottom of the liner 250. The top of the liner 250 is folded over the wire mesh support structure 100 (which is depicted in FIG. 8) to create a top fold 290.

In one embodiment, the liner 250 is a bag made from flexible, translucent material. Though light penetration through liner material is important, the material may be somewhat opaque in various embodiments. In one embodiment, material used for a liner 250 has light penetration as low as 50% and still provides sufficient light for photosynthesis to occur. In one embodiment, the liner 250 is made of low-density polyethylene (LDPE) or a similar material formed into a tubular shape and sealed at one end (FIG. 13). High-density polyethylene (HDPE), polypropylene, and various other materials may be used for the liner 250.

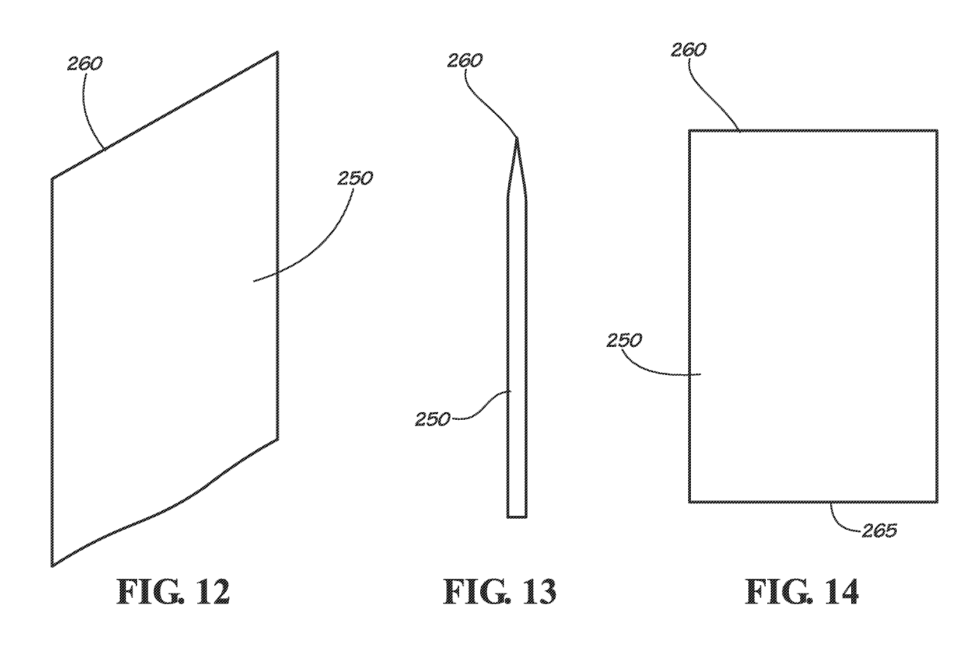

FIG. 11 shows the liner 250 that is inflated and sealed at one end maintaining its cylindrical shape with an opening on top as the liner 250 is folded to make a top fold 290 over the top of the wire support structure 100. Whereas FIG. 12 shows the liner 250 flat, before taking the shape of the support structure 100. In one embodiment, the plastic liner 250 is longer than the support structure 100 is high and heat sealed at one end making a sealed end 260 (FIG. 13). In one embodiment, the bottom corners 210 of the liner 250 are heat sealed at the bottom. (FIG. 16).

FIG. 14 shows the front view of an unsupported liner 250 with an opposing end 265 (from the sealed end 260).

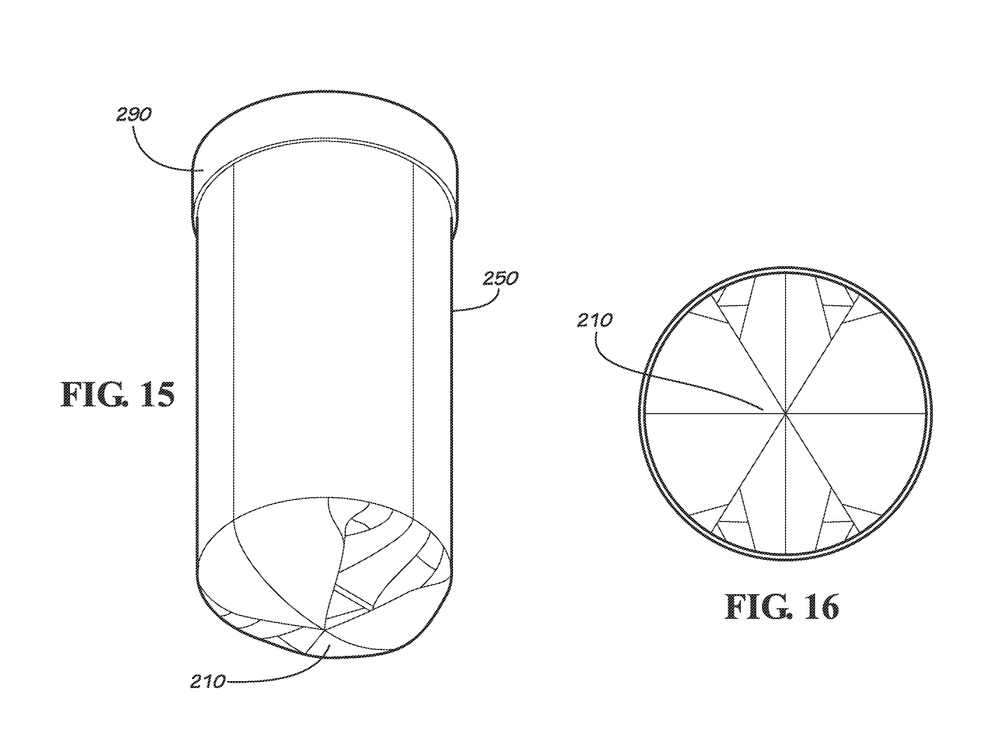

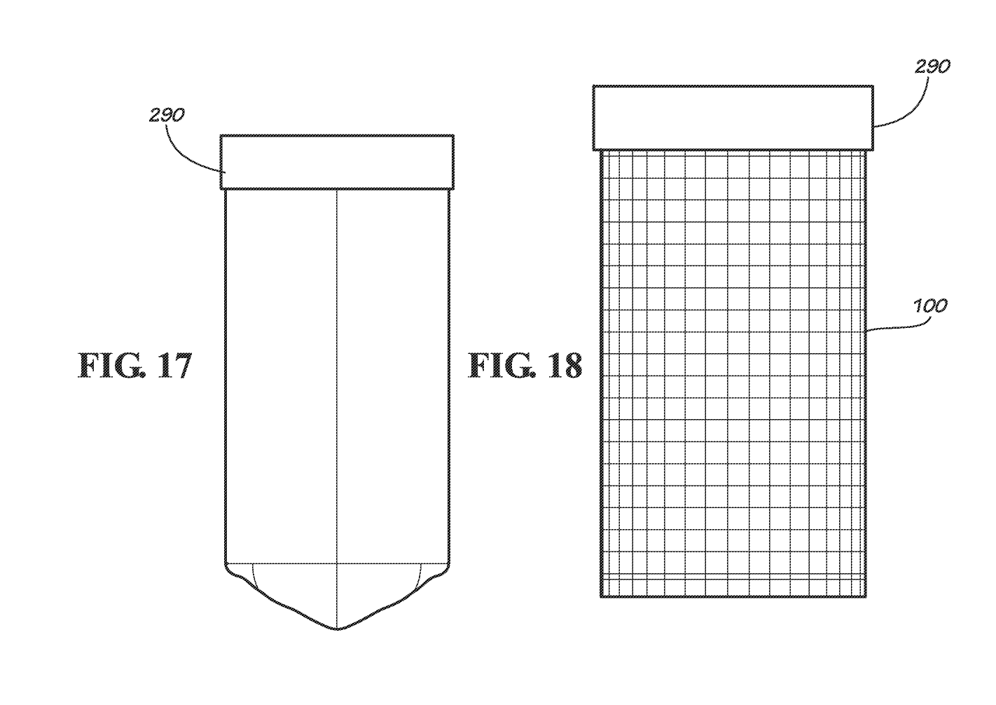

In one embodiment, the internal circumference of wire mesh support structure 100 is equal to the circumference of liner 250, enabling the liner 250 to be folded over the top of the support structure to make a top fold 290. (FIG. 15, FIG. 17). FIG. 15 also shows that the bottom corners 210 have been sealed on the bottom of the liner 250 (FIG. 16). FIG. 18 shows the liner 250 folded over the top of the support structure 100 making a top fold 290.

FIG. 18 shows a side view of the bioreactor module 050 with the top fold 290 of the liner 250 over the top of the wire mesh support structure 100. It also locates, but does not show that the bottom corners 210 that have been thermally sealed

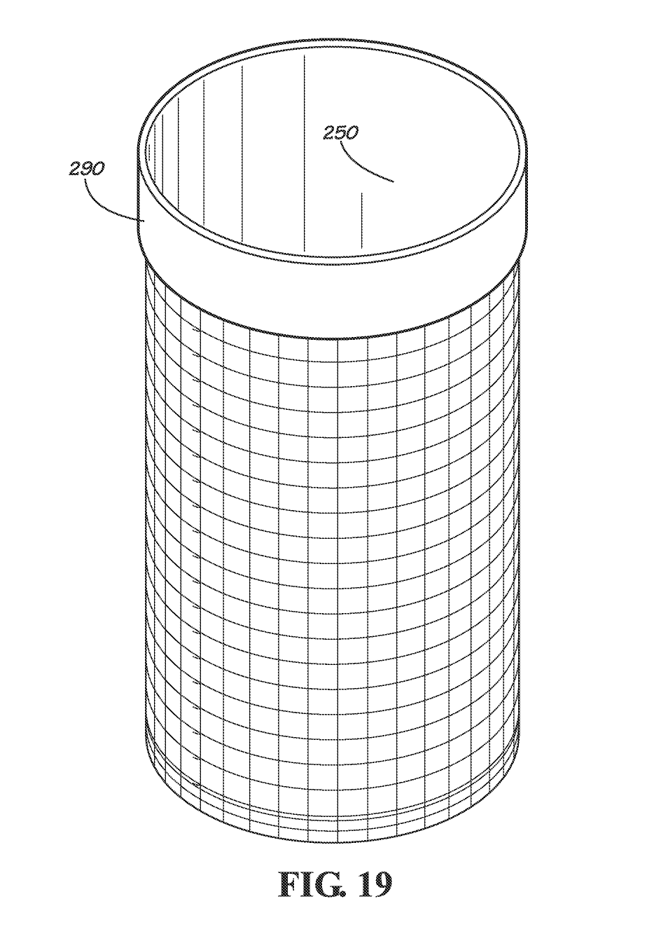

FIG. 19 shows a perspective view of the liner 250 inflated, within the wire mesh support 100 and the liner 250 is folded over the top to make a top fold 290.

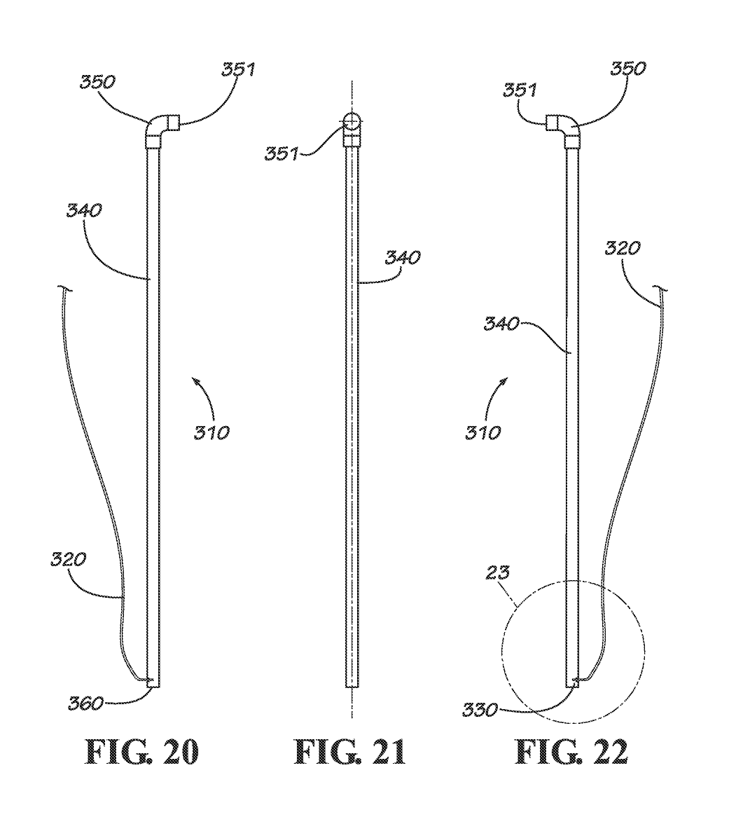

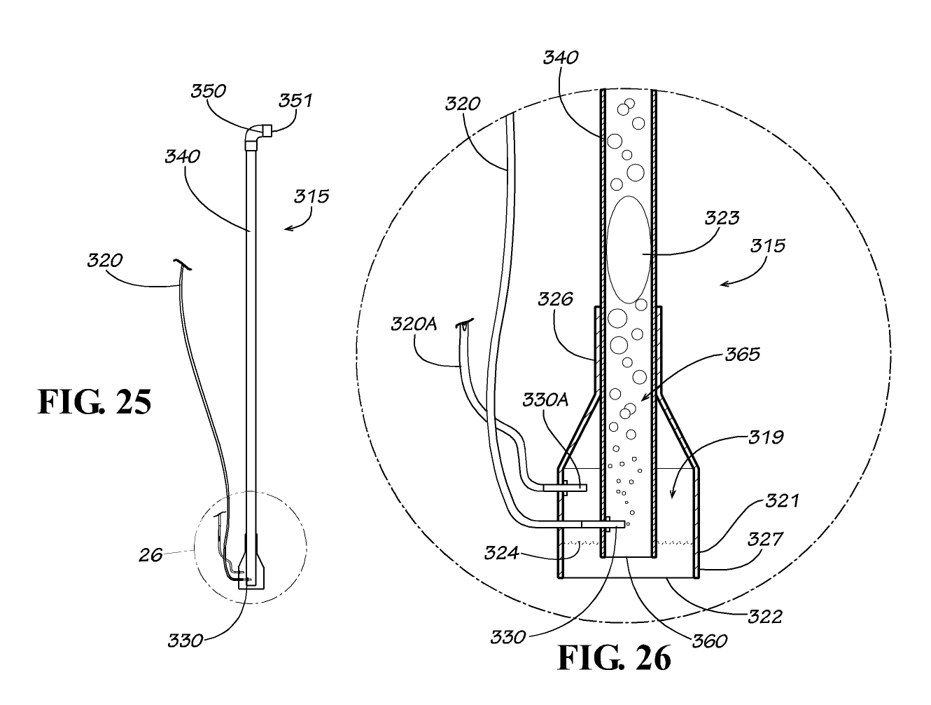

FIGS. 20-26 are embodiments of the air lift circulation and aeration pump 310

In one embodiment, the bioreactor module 050 has a rigid conduit or up-pipe 340 that creates an airlift pump 310 for transport of fluid and gas absorption. Gas is delivered to the bioreactor module 050 through a gas delivery line 320 and simultaneously mixes with the liquid solution and propels the solution vertically from the bottom of the bioreactor module 050 through the conduit 610 in the containing system 800 of interconnected bioreactors.

FIG. 20 is an embodiment of the air lift pump that is made of the up-pipe 340, the gas delivery line 320, and a fitting 350. FIG. 21 is a front view of the up-pipe 340 and an out-port 351. FIG. 22 shows that the gas delivery line 320 enters through a puncture in the bottom of the up-pipe 340. The fitting 350 is plastic welded in the current embodiment, or other equivalent seal, to the top of the up-pipe 340 redirecting the flow of solution 90 degrees. The out-port 351 from the fitting 350 is sized to press fit or permanently weld to an interior port 431 (as depicted in FIG. 30) of the liner/conduit fitting connector 440 (shown in FIG. 30). In one embodiment, the 90 degree fitting on the up pipe 340 and the fitting connector 440 is the same component.

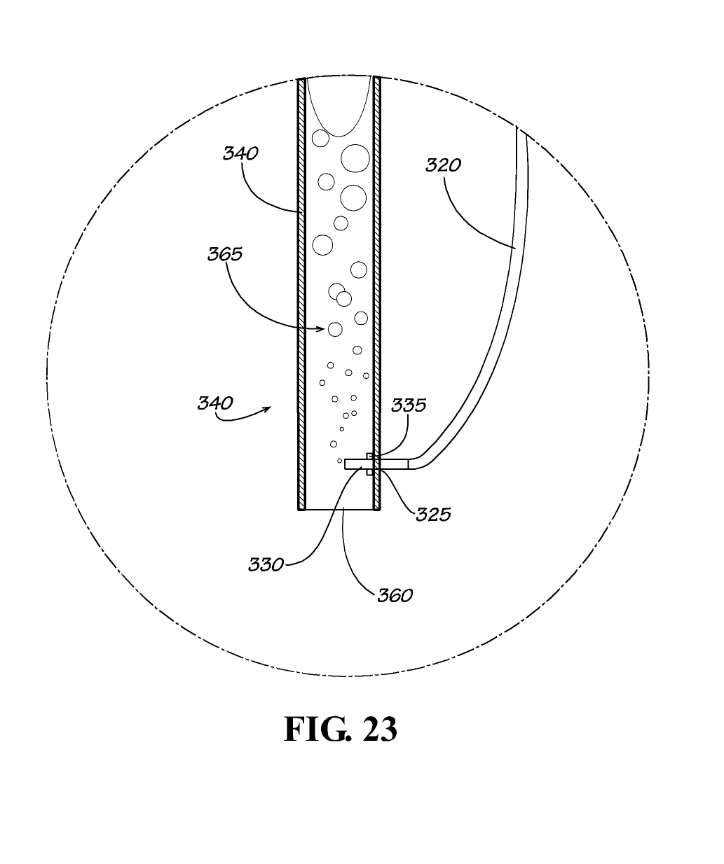

FIG. 23 is a detailed view of the bottom of the up-pipe 340 with gas intake tubing or gas delivery line 320 in use. At the base of each airlift conduit or up-pipe 340, gas is injected into the center of the up-pipe 340 via gas intake tubing or gas delivery line 320. This tubing or gas delivery line 320 connects permanently or by way of a press fitting 335 just above the base of the airlift conduit in the current embodiment. The flexible tubing passes through a puncture 325 in the liner 250 above the level of the solution, but below the top of the support structure. The puncture is smaller than the diameter of the tubing ensuring an airtight seal, although such a configuration is not necessary in every embodiment.

Intake tubing or gas delivery line 320 connects to system wide gas source outside of the bioreactor module 050 as shown in FIG. 45. In one embodiment, the system wide gas source includes a blower 710 that supplies pressurized gas through interconnected pipes 810 laid out to deliver equal pressure and flow to each bioreactor module 050 in the system. The blower intake may be filtered to reduce the risk of airborne contaminates, and the gas source can include air, carbon dioxide, and/or flue gas, or pressurized gas mixed to deliver optimal health characteristics for microorganism growth.

The gas delivery line 320 for the bioreactor module 050 in the current embodiment is made from standard aquarium tubing or equivalent flexible tubing that connects the gas injection port 330 to the gas delivery line 320. The gas delivery line 320 passes through a puncture in the liner 250 that is above the solution level and smaller than the diameter of the gas delivery line 320 forming an airtight seal. The airline then connects directly to PE gas delivery line 820 (as shown in FIG. 45) by way of standard press fit connectors.

The gas injector port 330 is made from rigid plastic or metal and serves the purpose of injecting gas into the center of the up-pipe 340 about one inch from the solution intake opening 360 of the air lift circulation and aeration pump 310. This fitting passes through a hole in the up-pipe 340 and the gas delivery line 320 and is press fit onto the fitting outside the up-pipe 340. This process is done so that the fitting is secured firmly in place and a seal is formed preventing leakage through the hole in the up-pipe 340 as depicted in FIG. 23. Silicon, epoxy or other adhesives may be used to strengthen the connection and seal.

In one embodiment, the up-pipe 340 is a vertical member made from PVC, PE or equivalent rigid pipe and serves the purpose of transporting gas/solution mixture vertically through the bioreactor module 050. The top of the up-pipe 340 is connected to a 90 degree fitting 350 that fastens to the liner/conduit fitting connector 440 enabling the transport of solution and gas to adjacent bioreactor module 050. The up-pipe 340 is cut to a length shorter than the overall bioreactor height so that the solution intake opening 360 resides 1-6 inches from the bottom of the bioreactor module 050 after air lift circulation and aeration pump is installed (FIG. 22). All up-pipes 340 used within the same containing system 800 of interconnected bioreactor modules 050 must be the same length to ensure equal performance between individual airlift circulation and aeration pumps 310 (FIG. 22).

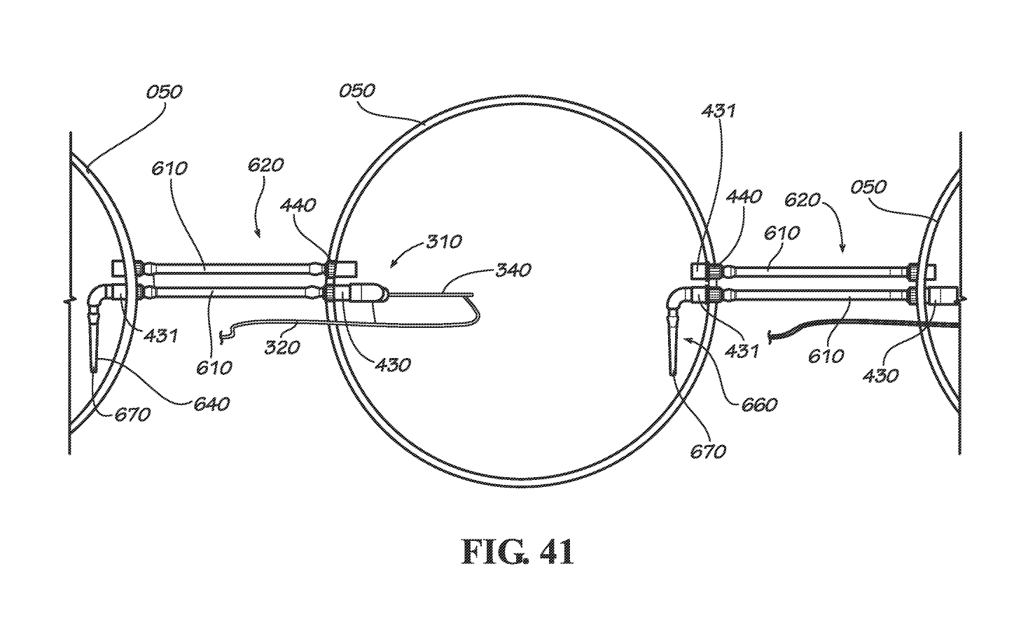

FIG. 23 shows injected gas forming small bubbles that rise upward, contained within the airlift circulation and aeration pump 310 and its components, gaining velocity on their way up. This process causes suction at the solution intake opening 360 and draws microorganism laden solution into the up-pipe 340. Buoyancy of the rising gas bubbles creates negative pressure in the up-pipe 340 and functions to lift the solution upward drawing it through the conduit 610 and solution injector 660 into an adjacent bioreactor module 050 (as shown in FIG. 41). In addition to transporting the solution, this process also functions to mix the gas with solution increasing the absorption rate of gas into solution over alternate aeration methods used for the cultivation of microorganisms.

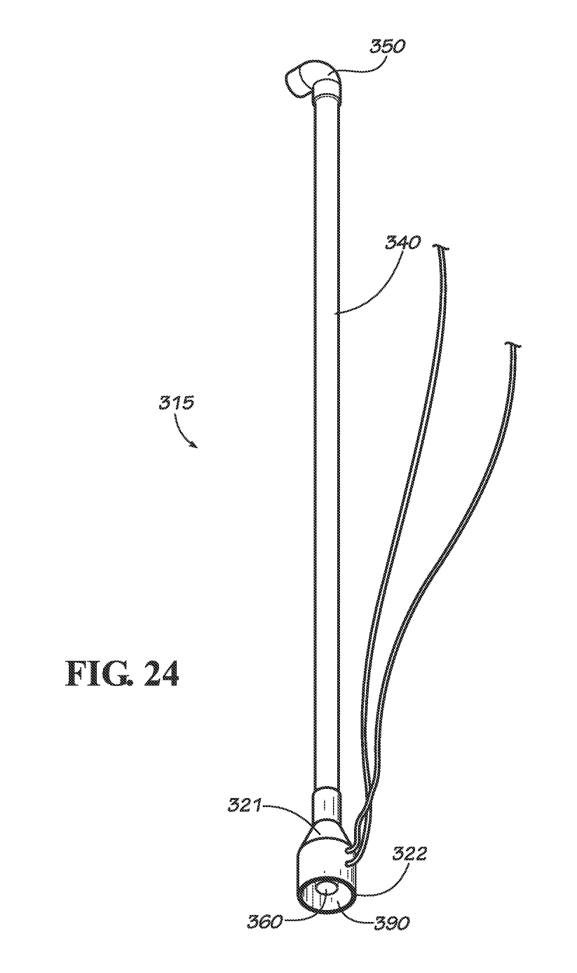

FIG. 24 shows one embodiment where the solution intake opening 360 at the bottom of the up-pipe 340 functions to draw solution containing live microorganisms into the bottom of the up-pipe 320. In one embodiment, a fitting may be used at the solution intake opening 360 of the up-pipe 340 to assist in vortex like circulation by creating tangential suction at the bottom of the bioreactor module 050.

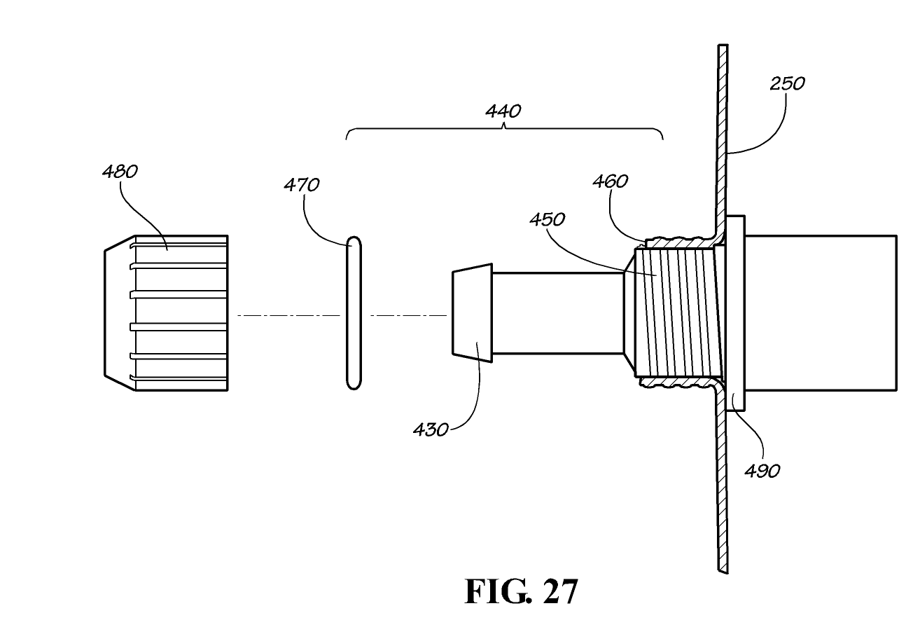

Another embodiment of an aeration pump 310 is an air lift geyser 315 as depicted in FIGS. 24-26. In some embodiments, this air lift circulation and aeration pump 310 may be used to increase efficiency and control over solution flow. Many of the components are the same as the aeration pump 310 previously disclosed; however, the addition of a secondary gas chamber 319 (as shown in FIG. 26.) provides additional lift making the pump more suitable for larger matter and increasing output pressure. In the same way as the standard air lift pump 310, gas is delivered to the bioreactor module 050 via gas delivery lines 320 that inject gas into air lift circulation and aeration pumps 310 and simultaneously mix gas with the liquid solution and propel the solution vertically from a location proximate the bottom of the bioreactor module 050 through the conduit 610 into the next bioreactor module 050 in the containing system 800.

In this embodiment, a second air line is used to deliver gas into the secondary gas chamber 319 (FIG. 26). The air lift/geyser circulation 315 and aeration pump 310 may also be used for harvesting purposes. Secondary gas chamber 319 is made using a standard PVC reducer 321 that is fastened by weld permanently onto the air lift up-pipe 340. In one embodiment, PVC reducer 321 is modified by the removal of material to allow for the up-pipe 340 to pass through the smaller end 326. In the current embodiment, the air lift up-pipe 340 passes through the small end 326 of the reducer 321 and is welded at the point where the up-pipe solution intake opening 360 is located approximately ½ inch vertically above the secondary solution intake opening 322. A secondary gas chamber 319 is created by attaching the reducer 321 in this way. This is because a cavity is formed between the inside of the large end 327 of the reducer 321 and creating the secondary gas chamber 319 (FIG. 26). An additional gas injector port 330A and gas delivery line 320A are fed directly into the secondary gas chamber 319. As the secondary gas chamber 319 fills with air, pressure builds and the volume of air pushes downward on the secondary gas chamber solution level 324. When this level reaches the up-pipe solution intake opening 360, a relatively large volume of gas 323 releases from the secondary gas chamber 319 into the up-pipe solution intake opening 360. Because the volume of gas 323 is larger than typical aeration in the up-pipe 340, it has higher buoyancy and accelerates vertically through the up-pipe 320, forcefully ejecting the contained solution through the conduit 610 into the adjacent bioreactor module 050. The solution is ejected in pulses. These pulses can be delayed by the time necessary for the pressure to build in the secondary gas chamber 319 to repeat the cycle. Between cycles, the standard gas injector port 330 is working to provide less forceful continuous flow and aeration. This function enables the control of solution flow with air pressure regulation, increases pump pressure, and further agitates solution leading to superior mixing and aeration.

In one embodiment, the bioreactor module 050 has a liner 250 and conduit fittings 440 as seen in FIG. 27. The fittings 440 are located above the solution level, but below the top of the support structure.

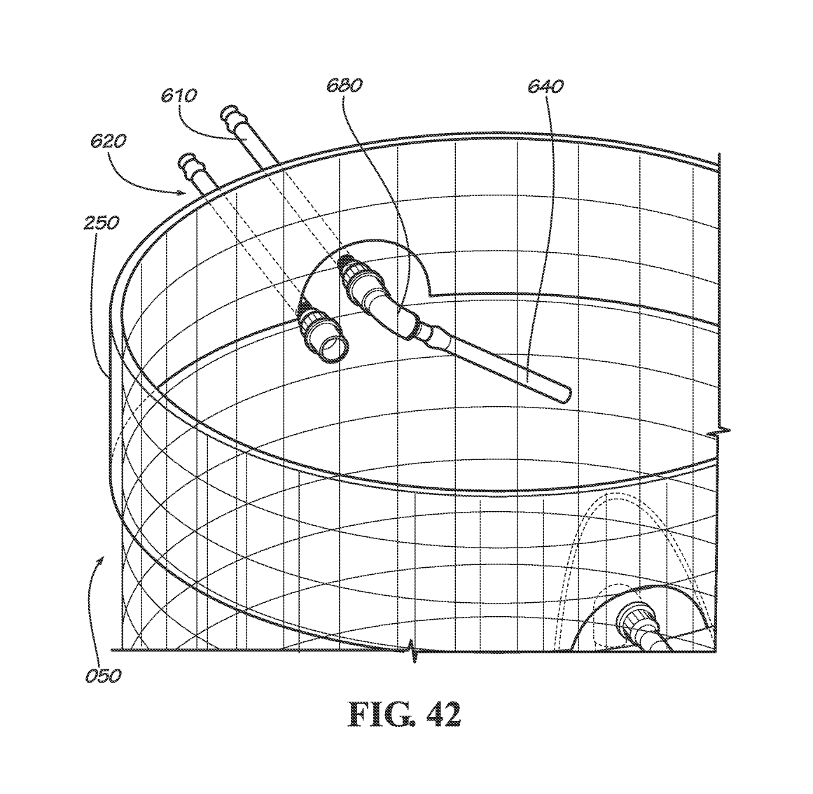

The interconnecting tubes 610 (as depicted in FIG. 42) enables fluid and gas transport to the adjacent bioreactor module 050. The liner 250 is punctured and the fittings 440 pass through the liner 250 via airtight fittings that seal the puncture in the bag against the fittings 440. These fittings 440 can be made from elastomeric materials, ridged plastic with elastomeric components, or threaded connectors 450, among other embodiments. The fittings 440 allow for an airtight seal and function to strengthen the liner 250 at the point of puncture.

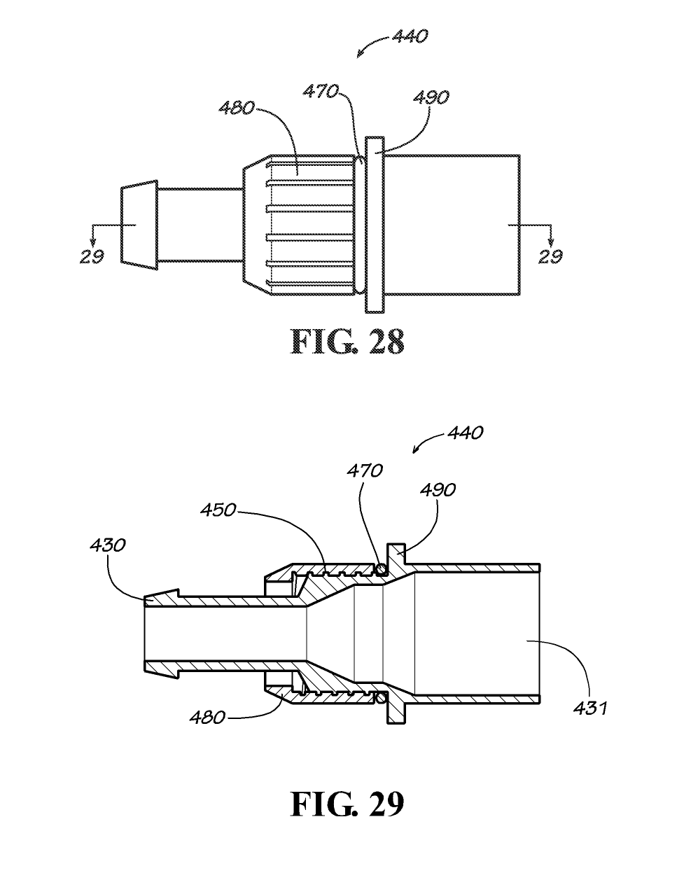

In another embodiment, above the solution level but below the top of the support structure the air lift circulation and aeration pump 310 bends 90 degrees redirecting solution and gas transport to the adjacent bioreactor module 050. At this level, the liner 250 is punctured with a pointed cylindrical tool one half to three fourths the diameter of the connection/seal threads 450 and the liner/conduit fitting connector seal threads 450 are pressed through the opening stretching the liner puncture 460 around the threads 450 creating a primary seal between the liner 250 and connector 440. The liner 250 is punctured and the threaded component is forced through opening in such a way that the plastic is in tension and stretched around the threads 450. An elastomeric O-ring 470 fits snugly over the stretched plastic liner opening 460 and connection/seal threads 450. A threaded nut 480 fits over the threads 450 and stretched plastic securing the O-ring 470 against the plastic and the connector flange 490 creating a secondary seal (FIG. 28).

In one embodiment, the threaded nut 480 is made of plastic and screws onto connection/seal threads 450 creating a primary seal. The nut is hand tightened compressing the O-ring 470 against the connector flange 490, which sits flush against the interior of the liner 250, pressing the liner between the plastic flange 490 and O-ring 470 creating a secondary seal that removes stress from the primary seal and helps to stabilize connector 440 with liner 250 (FIG. 27, FIG. 28). The press fit connector outlet 430 reduces the inner diameter of the conduit 610, increases pressure, and allows for the connection of PE connector conduit 610, which facilitates solution flow from one bioreactor module 050 to the next within a containing system 800 of interconnected bioreactor modules 050.

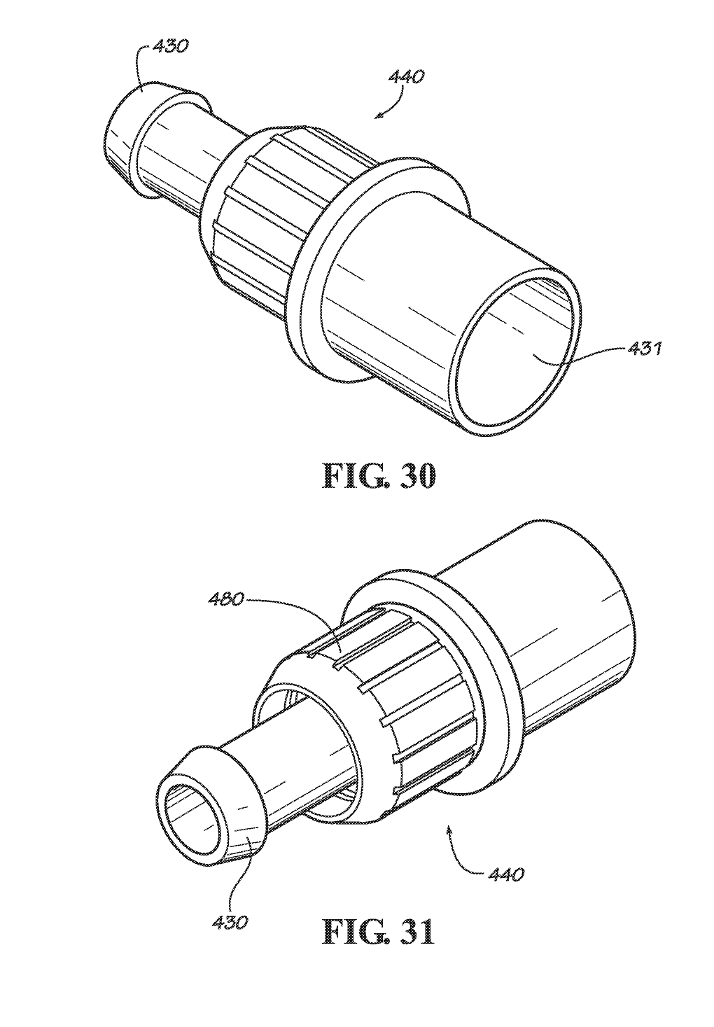

FIG. 29 shows the interior port 431, of a fitting that is fitted to connect directly to the air lift 90 degree fitting 350 functioning as a solution inlet. The interior port 431 may also connected directly to the solution injector 660 in the adjacent bag and may function as an outlet as depicted in FIG. 41. The liner/conduit fitting connector 440 is also used for overflow redundancy pipes or overflow conduit 620 (pictured in FIG. 41). In this embodiment, the interior port 431 is left open to accept flow in either direction preventing overflow.

Alternate embodiments of these fittings can be made from elastomeric materials, ridged plastic with elastomeric components, or threaded connectors 450. The fittings allow for an airtight seal and function to strengthen the liner 250 at the point of puncture. Current configurations use a combination of the aforementioned options.

FIGS. 30 and 31 show perspective views of the conduit fitting connectors 440. FIG. 30 shows the interior port 431, press fit connector outlet 430, and the threaded plastic nut 480. FIG. 31 shows the press fit connector outlet 430 and the threaded plastic nut 480.

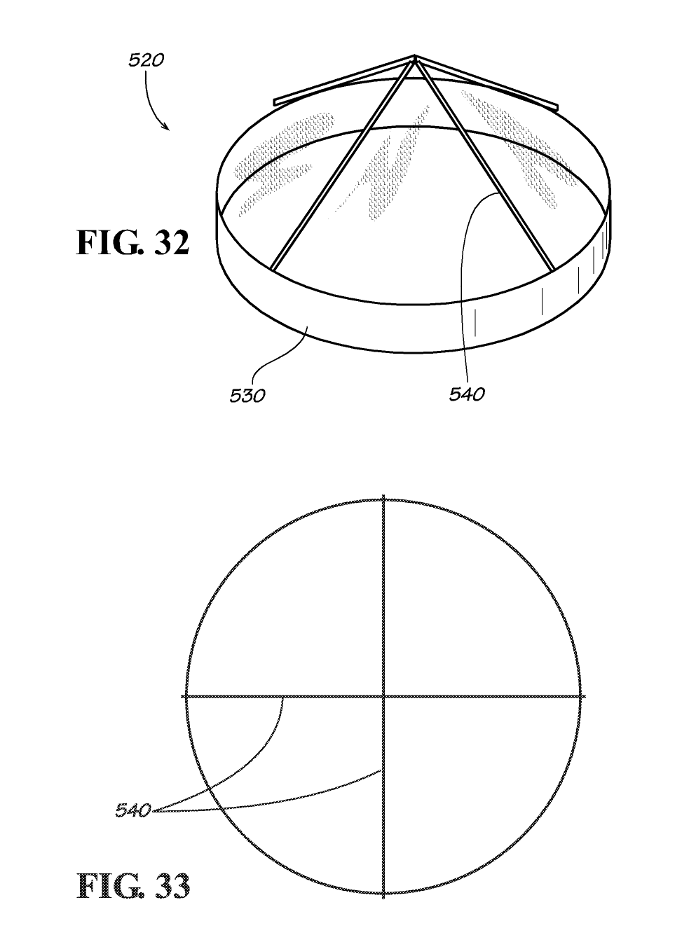

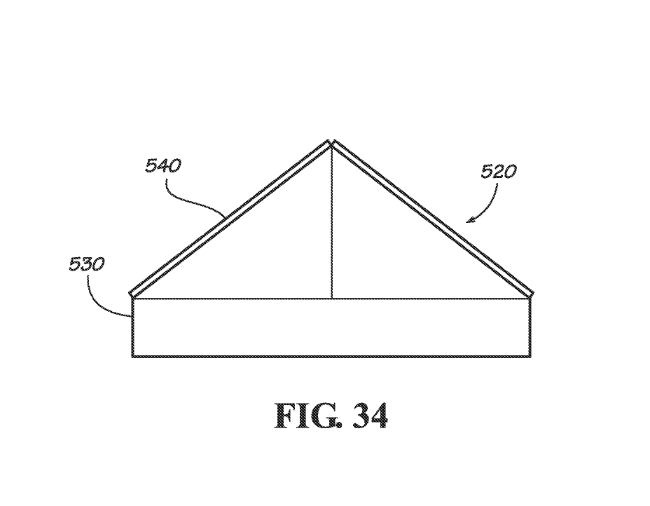

In order to close the system, prevent overflow and dilution from rainwater, and prevent contamination—as would be found in an open pond system—the bioreactor module 050 is covered in the current embodiment. A cap 520 is made from a transparent material and prevents the intrusion of contaminants (FIGS. 32-34). The cover 520 will fit securely over the overlapped top fold 290 of the liner 250. (FIGS. 17 and 18).

In one embodiment, the cover 520 includes a transparent material that fits tightly over the bioreactor module 050 preventing contaminates from getting in. In this embodiment, the cover 520 is configured by using the same material as the transparent flexible liner 250. The liner 250 is cut to a shorter length and sealed on one end in such a way that the cover 520 can be stretched over the top of the bioreactor module 050 and the positive pressure from the gas input keeps the cover 520 inflated giving it form and helping prevent collapse. (FIG. 33)

The cover 520 fits snugly over the bioreactor module 050 creating a plastic-on-plastic seal. The cover 520 fits tightly enough to prevent contaminates from entering into the system but allows for the escape of excess pressure. In the event that the cover 520 fits too tightly over the bioreactor module 050 and positive pressure becomes too great, a small diameter tube can be fitted between the bioreactor module 050 and cover 520 allowing the escape of excess gas in some embodiments. The sidewall 530 of the cover 520 extends over the wire mesh support 100 structure and top fold 290 of the liner 250 creating a plastic on plastic seal. In the event that the cover fits too loosely over the bioreactor module 050, grommets may be used or holes may be cut allowing the cover to be fastened by way of tying or zip tie. Tape or other means may also be used to fasten cover 520 to wire mesh support structure 100 more permanently.

FIGS. 32-33 show a thermally welded seam 540 formed by laying the cut liner material flat and using a bag sealer to seal the liner at an angle from both sides so that the seal meets below the center of one open end. This forms a “V” shape with the point residing in the center of the liner material and the ends extending through the edge of the liner material 10-20 cm above the open end. The liner material is then opened, rotated 90 degrees, and pressed flat so that an additional seal can be made of the same proportions as the first resulting in 4 angular seals terminating at one point in the center of the liner 250.

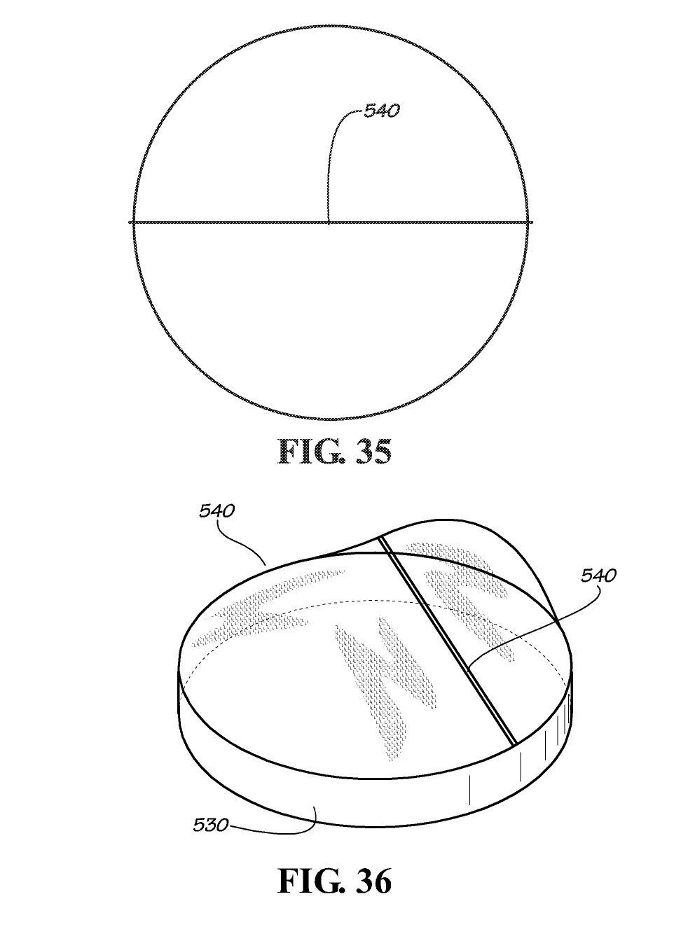

In another embodiment, the bioreactor cover 520 is a simpler variation of the first, whereby only the first of the aforementioned seams 540 are made. (FIG. 35). This configuration requires significantly less labor, but requires more positive pressure to stay inflated and resistant to the elements. FIG. 36 shows a cover with one seam. Other cover variations and materials may be used to achieve similar results.

Different bioreactor modules 050 may require different gases as well as different nutrients depending on the microbes growing and what they require. In one embodiment, the bioreactor modules 050 are supplied carbon dioxide from the gas delivery lines 320. In another embodiment, nitrous gases are delivered through the gas delivery lines 320. Many embodiments of this disclosure allow the bioreactor modules 050 to take many different types of pollutants, gasses, or various other media into the bioreactor modules 050.

The bioreactor module 050 is designed to stir or mix the media, so the diameter of the support structure 100 structure can vary. While light is a limiter to microbial growth, it can be overcome with good mixing. Good mixing allows each microbe to get light exposure from the turbulent mixing in the system. This is an advantage of the present system: it mixes both within each individual bioreactor module 050 and mixes between each bioreactor module 050 as the microbes flow through the entire multi-reactor containing system 800 as they are connected. This is an uncommon advantage.

In one embodiment, the bioreactor modules 050 are connected by a PE connector conduit or interconnection tubes 610 as shown in FIG. 41. The interconnection tubes 610 link the airlift conduit or up-pipe 340 to the solution injector 660 in the next bioreactor module 050. Interconnection tubes 610 connect to the fitting components linking the bioreactor modules 050 via the air lift conduit or up-pipes 340 on one side and solution injector 660 in the next bioreactor module 050 in the closed loop of the containing system 800 of interconnected bioreactor modules 050.

The interconnection tubes 610 may also be utilized as overflow protection, as shown in FIG. 41. In this case, an additional overflow conduit 620 connects bioreactor modules 050 with fitting components, but on the inside of the bioreactor module 050 the fitting terminates with only an opening in the interior port 431. This allows solution flow between interconnected bioreactor modules 050 within the same containing system 800 in the case of uneven airlift pump performance or failure (FIG. 41). This feature ensures even fluid levels between all bioreactor modules 050 in a given containing system 800 of interconnected bioreactors. Interconnection tubes 610 can be PVC, PE, vinyl or equivalent tubing.

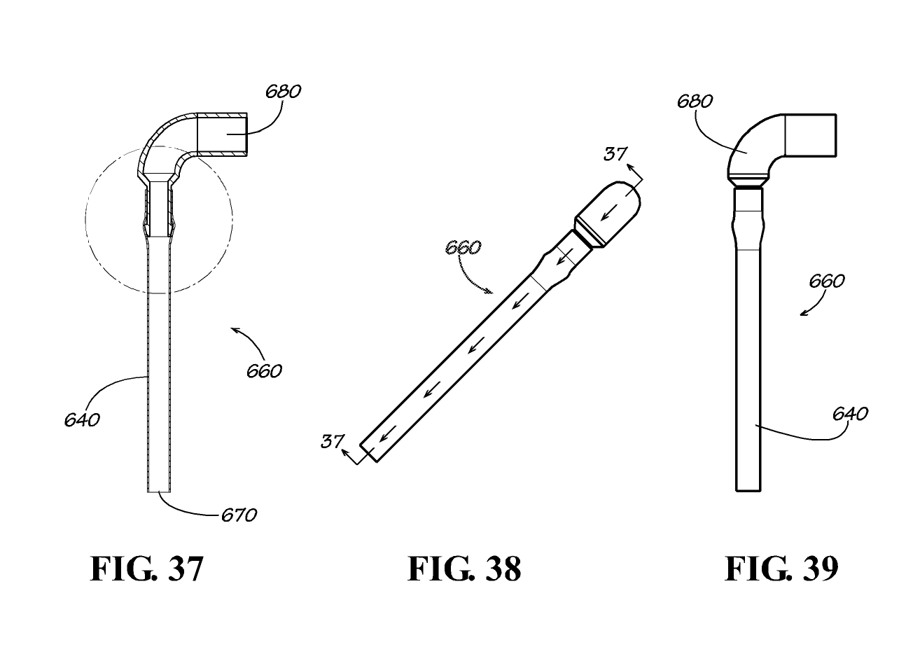

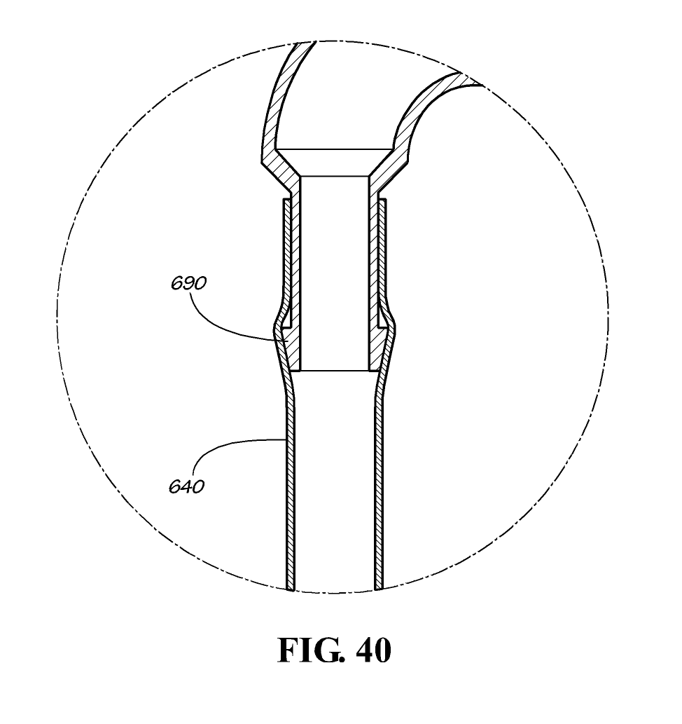

FIG. 37 shows the injector tube 640 and the 90 degree fitting 680 of the injector port 660. FIG. 38 shows the injector port. The arrows show the solution flow through the tubing. FIG. 39 shows the 90 degree fitting 680 of the injector port 660 and the injector tube 640. In one embodiment, the injector tube 640 is made from PE tubing, or other material in the same or lesser diameter as the connection conduit 610 (as depicted in FIG. 42), and is press fit onto the 90 degree fitting press fit connector 690 (seen in FIG. 40). The injector port 660 is connected 90 degrees relative to the horizontal interconnection tubes 610. In this position, the solution would flow straight downwards. However, in other embodiments, the solution injector 660 may inject solution at 10-45 degrees, relative to the vertical support structure 100 as depicted in FIG. 42), promoting a circular stirring effect within the bioreactor module 050.

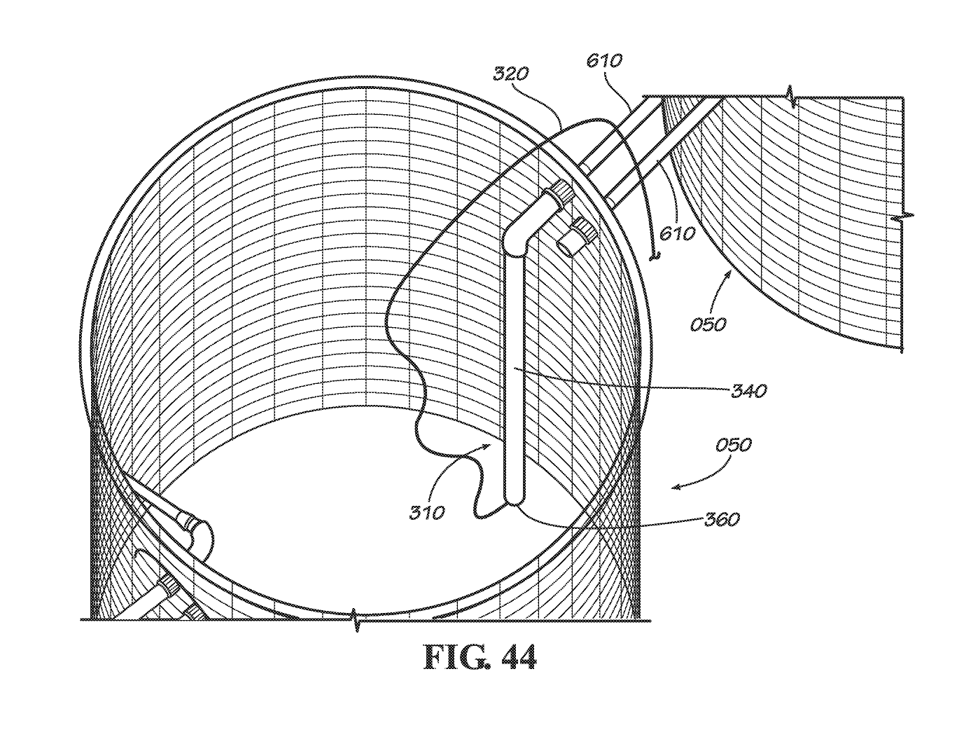

Generally, gas and liquid solution will be pumped through the air lift circulation and aeration pump/up-pipe 340, and be transported through the fittings 440 and connection conduit 610 into adjacent bioreactor module 050 as can be seen in FIG. 41 and FIG. 44. On the interior of the bioreactor module 050, a solution injector 660 is fastened by press fit or weld to the fitting connector interior port 431 (FIG. 41). This is done so that the solution injector exit port 670 is at a slight downward angle and injects solution tangentially to the liner circumference (FIG. 42). This configuration is intended to produce a stirring effect where the bioreactor module 050 contents are continually being mixed in a gentle vortex like manner.

FIG. 41 shows that individual bioreactor modules 050 may be connected in series by interconnection tubes 610 that link airlift circulation and aeration pumps 310 to solution injector 660 in the next bioreactor module 050. Interconnection tubes 610 connect to the liner/conduit fitting connectors 440 by way of a press fit connector 430 linking the bioreactor modules 050 with the air lift circulation and aeration pump 310 on one side and solution injector 660 in the next bioreactor module 050. This linkage facilitates solution flow from one bioreactor module 050 to the next within the containing system 800 of interconnected bioreactor modules 050.

In one embodiment, solution may be injected out of the solution injector port 670 into the top of the bioreactor module 050 just above or below the liquid level, depending on depth and angle that creates a gentle vortex stirring effect. The 90 degree fitting 680 connects to the fitting connector interior port 431 on one side via a connector that may be press fit or welded to the fitting connector 440, and on the other it connects to the PE injector tube 640 via a press fit or welded connection.

Interconnection tubes 610 may also be utilized as overflow protection. In this case (FIG. 42), an additional connector conduit operates as an overflow conduit 620 connects the bioreactor modules 050 with fitting components, but on the inside of the bioreactor module 050 the fitting terminates at the interior port with only an opening at the interior port 431 that allows for solution flow between bioreactor modules 050 in the case of uneven airlift pump performance or failure. This feature ensures uniform fluid levels between all bioreactor modules 050 interconnected in a given containing system 800.

Interconnection tubes 610 can be PVC, PE, vinyl, stainless steel, aluminum, copper or equivalent tubing, among other embodiments. The interconnection tubes 610 functions to connect bioreactor modules 050 and enables fluid flow from the air lift circulation and aeration pump 310 to the solution injector 660. This overflow conduit 620 is also used for over flow redundancy preventing bioreactor module 050 overflow in the event of a singular pump failure or inefficiency.

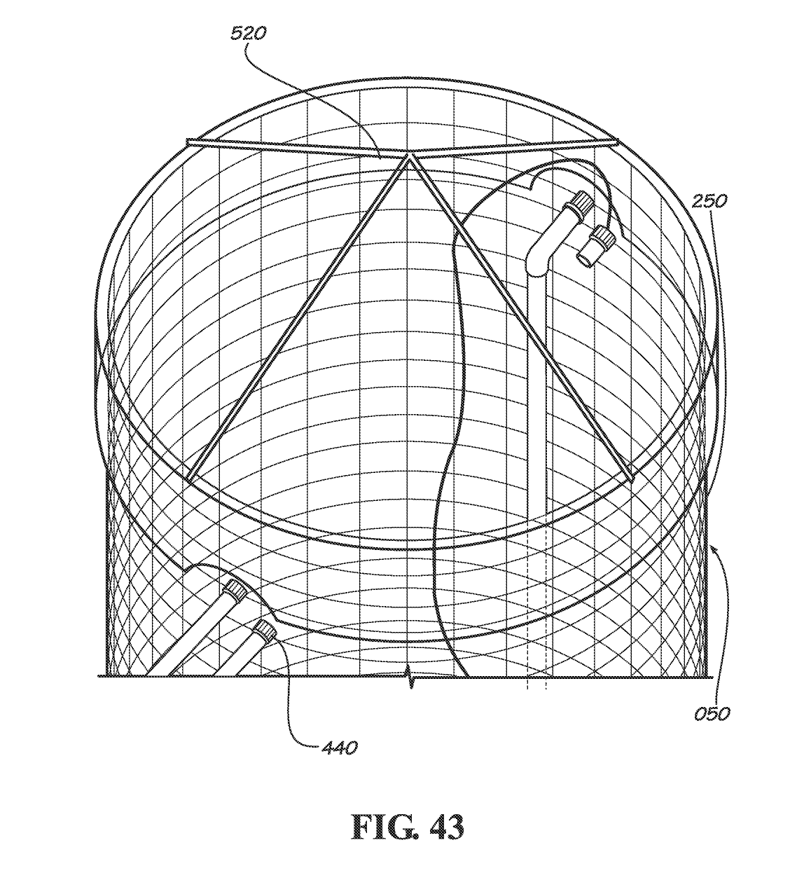

FIG. 43 shows a perspective view of the bioreactor module 050 including the inside of the bioreactor module 050, the liner 250. It also depicts the outside connections including the fitting connector 440.

FIG. 44 shows the up-pipe 340 of the air lift pump 310 on the inside of the bioreactor module 050. It also shows the solution intake opening 360 and the gas delivery line 320 at the bottom end of the up-pipe 340. The top of the up-pipe 340 is connected to the interconnection tubes 610.

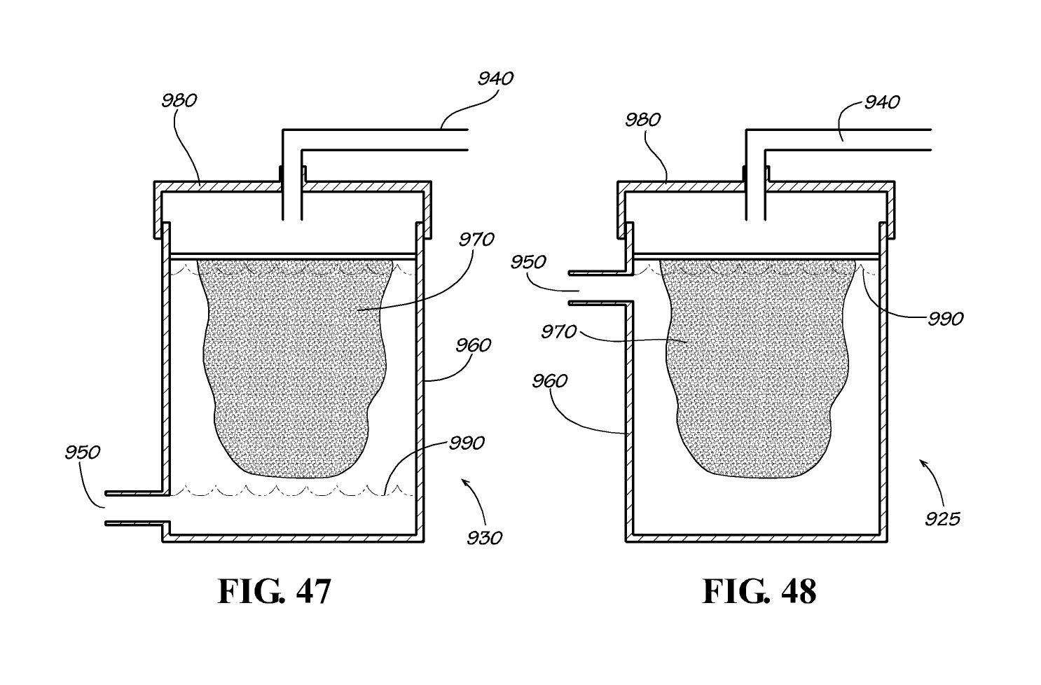

In one embodiment, the bioreactor module 050 is one module. In another embodiment, there is a containing system 800 of multiple interconnected bioreactor modules 050, as seen with reference to FIG. 45. The overall bioreactor system 8000 uses the same gas delivery system. Some bioreactor systems have multiple containing systems 800 of interconnected bioreactors. A containing system 800 is a smaller bioreactor system, made of multiple interconnected bioreactor modules 050. The bioreactor modules 050 in the containing system 800 may all attach and share the same harvesting unit 900 through a harvesting port 910 (shown in FIG. 47-49 and water return inlet 920. In one embodiment, a bioreactor system is made of multiple containing systems 800.

In one embodiment, a gas delivery system 805, as depicted in FIG. 45, may include one or more high pressure industrial blowers 710 with working pressure of at least 120 mbar for a nominal solution level of 1 meter. Other variations of this embodiment are also possible. This pressure is sufficient to deliver gas to the bottom of the bioreactor module 050 overcoming water pressure at a depth of 1 meter. For alternate configurations such as shortened bioreactor height, working air pressure requirement will change depending on the pressure at depth of solution. Air intake to the blower 710 is filtered through an air filter 730 to remove large particles that may clog the microbial air filter 740. The blower 710 is connected directly to the gas delivery backbone 810 made of metal and/or PVC pipe that is sized according to total number of bioreactor modules 050 enabling equal pressure gas delivery, although particular features of the design may change from one embodiment to another. In one embodiment, the blower 710 is the driving force providing gas flow through the containing system 800. In another embodiment, gas enters the containing system 800 simply through an exhaust, air vent, or other embodiments. These embodiments cause the gas to flow through the containing system 800.

The bioreactor modules 050 may be organized into containing systems 800 that share harvesting units 900 and water return inlets 920. FIG. 45 shows a bioreactor system with three containing systems 800A,B,C. The central containing system (“B”) has fourteen bioreactor modules 050, a gas delivery line 820B, a harvesting port 910B, a harvesting unit 900 and a water return inlet 920B. The more individual bioreactor modules 050 that are interconnected sharing one harvesting outlet 910, the more production for that harvesting outlet 910. This also allows for more production of algae and consumption of carbon dioxide per containing system 800, which in turn reduces costs and labor associated with set up, harvesting, and maintenance.

The gas delivery backbone 810 may be made of stainless steel, Galvanized steel, polypropylene, and/or PVC pipe sized according to air flow requirements that vary based on overall system size (number of individual bioreactor modules 050 connected to air delivery system). Material specification is dependent on gas temperature, and portions of the gas delivery backbone 810 may be made from varying materials.

The gas delivery system has a gas delivery backbone 810 that delivers the gas to the gas delivery line(s) 820 of the individual containing system(s) 800. In one embodiment, the gas delivery line 820 is PE hose sized depending on number of individual bioreactor modules 050 in each containing system. Most configurations use 2 cm PE hose. PE hose enables the use of press fit connectors that significantly reduce labor and maintenance costs. Bioreactor gas delivery lines 320 connect to the containing system 800 gas delivery line 820 with commercially available connectors traditionally used for drip system irrigation. Other materials other than PE may be used to achieve a similar purpose.

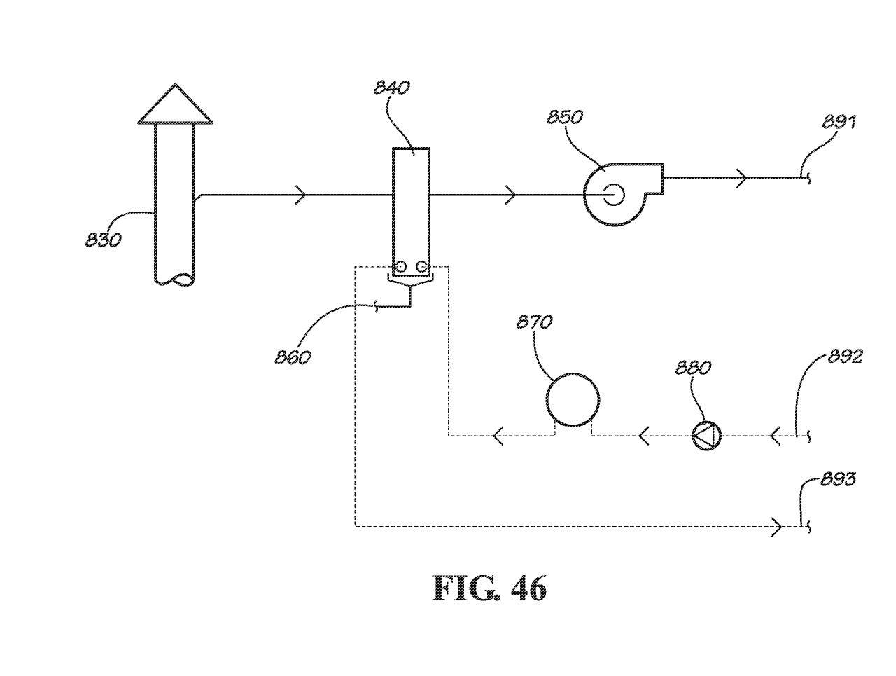

The bioreactor system may have many embodiments. In one schematic layout, as depicted in FIG. 46, the bioreactor module 050 system includes any number of individual bioreactor modules 050. Individual bioreactor modules 050, each having air lift circulation and aeration pumps 310, are joined into continuous interconnected loops by connecting interconnection tubes 610 from one bioreactor module 050 to the next. This creates even solution circulation among all joined bioreactor modules 050 within the containing system 800. The containing system 800 of joined bioreactor modules 050, in effect, functions as one unit and the containing system 800 of interconnected bioreactor modules may be harvested as such. Due to the even flow in the system, nutrients, microbial starters, and other necessary additives can be added to a bioreactor module 050 at one point and will be distributed evenly throughout the containing system 800 over a period of time allowing circulation to occur.

After the bioreactor module 050 and the system are assembled, the initial water source for the system is sterile. Sterilization techniques include, but are not limited to, ozone, UV light, chlorination and de-chlorination, and/or filtration.

System wide sterilization using ozone gas utilizes ozone gas fed through the system wide gas pipes, or into individual bioreactor modules 050 via the gas delivery lines 820. The ozone gas is supplied with sufficient pressure to operate the airlift pumps and run the system with only water in it. In this case, the bioreactor modules 050 are filled with non-sterilized water, and the ozone gas runs the system sterilizing the water, gas lines, pipes, fittings, and bioreactor material. Upon successful sterilization, filtered air is pumped through the sterilized system flushing the residual ozone which prepares the system for the addition of algae or microorganism starter.

In one embodiment, one bioreactor module 050 is used to cultivate microorganisms. The basic bioreactor design can easily be run as a single unit. In this embodiment, air stones may be used to mix the media instead of the airlift circulation and aeration pump 310 and solution injector 660. The medium does not need to flow. In one embodiment, multiple bioreactor modules 050 are assembled together to form an interconnected containing system 800 of bioreactor modules 050. The flow of media from one bioreactor module 050 to another improves mixing and also allows automation in harvesting, as well as providing ease of maintenance to add supplemental nutrients.

In one embodiment, a containing system 800 is comprised of 10-30 interconnected individual bioreactor modules 050. In one embodiment, where the microorganism is algae, a containing system 800 of this size should yield a harvest of 5 to 50 kgs/week, depending on the size of the bioreactor modules 050 and other variables.

The bioreactor module 050 of present disclosure can be used to cultivate various types of microorganisms. The bioreactor design and the sterilization system are microorganism independent. In one embodiment, algae are cultivated. In another embodiment, Spirulina are cultivated. In yet another embodiment, Chlorella are cultivated. While the bioreactor system is independent of the type of organism used, the harvesting system module may be modified depending on the size of the microorganism.

The system of the current disclosure is effective for cultivating other more sensitive species. For example, many of the species grown for oil production are very sensitive and require a closed system. The system of the current disclosure is designed and intended to provide the control necessary to cultivate such species at a fraction of the cost.

Inoculation of the bioreactor containing system 800 can be done by introducing the microorganism culture into a sterilized system through one port (harvester outlet) 910 and then the system distributes the inoculants equally to the individual bioreactor modules 050 interconnected in that containing system 800. Other systems would require each bioreactor module 050 to be inoculated individually. Nutrients and additional water may be added in the same manner.

In one embodiment, prior to inoculation into bioreactor module 050 or the containing system 800, the microorganisms are acclimated to the bioreactor environment and gaseous carbon dioxide injection in a lab. Growing the initial culture in the lab with the same environment will give rise to a natural selection of microorganism. This means that the organisms that are eventually used to inoculate the outdoor system which will be hardier and grow better with gaseous carbon dioxide as a major part of their inorganic carbon supply.

As the bioreactor system includes multiple bioreactor modules 050 connected into a continuous loop forming a containing system 800, harvesting may be achieved between any two bioreactor modules 050 in a given containing system 800 by directing solution flow through a harvesting unit, or by way of separate harvesting pumps and connectors. For the purpose of clarity, the harvesting outlet 910 resides on the last bioreactor in the system of the current embodiment, and the water return inlet 920 resides on the first bioreactor in the system of the current embodiment, although such a configuration is not necessary for proper functioning of the system. (FIG. 45).

In one embodiment, the bioreactor harvesting outlet 910 is a permanent connector in the last bioreactor module 050 in the system. The outlet is made of a liner conduit fitting connector 440 that is connected permanently to a valve 955 outside the bioreactor module 050 enabling the port to be opened or closed. Inside the last bioreactor module 050, an additional air lift circulation and aeration pump is connected to the bioreactor harvesting outlet 910. The gas delivery line 320 to harvesting air lift pump is fitted with a flow control valve outside the bioreactor module 050 allowing the pump to be disabled when not in use.

A post harvesting water return inlet 920 resides on the first bioreactor module 050, or the bioreactor module 050 following the one containing the harvesting outlet 910, and functions as a return port for solution that has passed through the harvesting unit. The post harvesting water return inlet 920 is a permanent connector on the first bioreactor module 050 in the system. The harvesting outlet 910 is made of a liner conduit fitting connector 440 that is connected permanently to the first bioreactor module 050 enabling the return of solution into the system after harvesting. In addition to functioning as the return inlet for harvesting unit, water and nutrients may be added to the closed system through this port. Water is returned to the system after harvesting lowering water consumption, and returning nutrients still present in solution and returning immature (still growing) microbial cells that are small enough to pass through the harvesting sieve 970.

FIG. 47 shows one embodiment of the fixed-time harvester 930 that consists of a rigid harvesting vessel 960 which houses a harvesting sieve 970, a solution inlet 940, and clear solution harvester outlet 950 back to bioreactor module 050. The harvesting sieve 970 may be any material suitable for filtering pre-determined particulate size from solution. In one embodiment, a polyester cloth may be used as a filter/bag that collects microorganisms while allowing water to pass through. The harvesting sieve 970 is attached to the upper portion of the harvesting unit 900 and is in a position to capture or filter the solution and media that enters the rigid harvesting vessel 960 from the solution inlet 940.

The harvesting unit 900 may be connected either temporarily by way of press fit connectors 440, which may be threaded connections or permanent welded connections, to the bioreactor harvesting outlet 910 or the post harvesting bioreactor water return inlet 920. Connections may be made with any form of suitable pipe or hose conduit, including but not limited to, PVC, polyethylene, or vinyl. The harvesting unit sits atop a permanent or moveable stand at such a height where the harvester outlet 950 is horizontally in line with or vertically higher than the bioreactor water return inlet 920 enabling overflow back into the bioreactor module 050 without the use of additional pump. This height ensures that any failure in the harvester will not affect the flow of the system or cause spillage outside the bioreactor module 050. In some embodiments, a pump may be included, obviating any specific geometric orientation.

In one embodiment, as depicted in FIG. 47, the harvesting is done for a fixed period of time. The fixed-time harvester 930 is connected to a bioreactor module 050 so that the containing structure drains filtered solution from the bottom into the next bioreactor module 050. In one embodiment, the filtration media is hung from the top of the rigid harvesting vessel 960. Solution containing microorganisms is pumped from the last bioreactor module 050 into the middle of the filtration media contained within fixed-time harvester 930 containing structure (“last” is used to define the output bioreactor). Water passes through the filtration media and drains from the containing structure via a connection hose into the first bioreactor module 050 (“first” is used to identify the bioreactor succeeding the “last” bioreactor module 050). In this embodiment, microorganisms trapped in the filtration media can be manually removed from a module. The quantity of harvest is determined by the time that the harvester is left connected and running.

The harvester inlet 940 connects to the bioreactor harvesting outlet 910 via a connecting hose, and delivers microorganism laden solution into the middle of the harvesting sieve 970. The harvester outlet 910 connects to the bioreactor water inlet 920 returning the filtered solution back into the containing system 800 of bioreactor modules 050. The rigid harvesting vessel 960 can be made from any material. In the current embodiment, the unit is continuously opened and closed to harvest and is made from a material that can be easily sterilized.

The harvesting sieve 970 functions as a filter allowing water and very small particles to flow through while trapping grown microbial cells for harvest. This sieve is removable from the harvesting unit. At the time of harvest, sieve 970 is removed from unit and emptied into a collection vessel. In the case of continuous harvesting, the sieve 970 may be removed and replaced immediately with a like empty part.

The harvester lid 980 functions to keep the system closed and prevent contamination.

The solution level 990 varies within the harvesting outlet 910 depending upon whether the unit is used for a fixed period of time or continuously. Depending on where the harvester outlet 950 resides on the harvester, solution level 990 inside the harvesting outlet 910 may be below the sieve 970 entirely for fixed time harvesting or the sieve 970 may be suspended in solution, as in the case of continuous harvesting.

In another embodiment, as depicted in FIG. 48, the continuous harvester 900 harvests microbes continuously. Continuous harvesting works in much the same way as fixed-time harvesting; however, the harvest size is limited by the size of the filtration media, and rather than draining water from the bottom of the harvester vessel, the harvester is allowed to fill to near the top of the filtration in such a way that the solution level 990 keeps the harvested microorganisms submerged and alive for extended periods of time. By this method (FIG. 48), labor required for harvesting may be significantly reduced. When the harvesting sieve 970 fills completely with densely packed algae cells, solution will overflow into the rigid harvesting vessel 960 and flow through the harvester outlet 950 back into the system. Because of this redundancy, the precise time at which the harvesting takes place is not important. Additionally, regulating solution flow into the filtration media in such a way that the quantity of harvested microorganism may be predetermined over a period of time may also be employed to achieve desired harvest quantity.

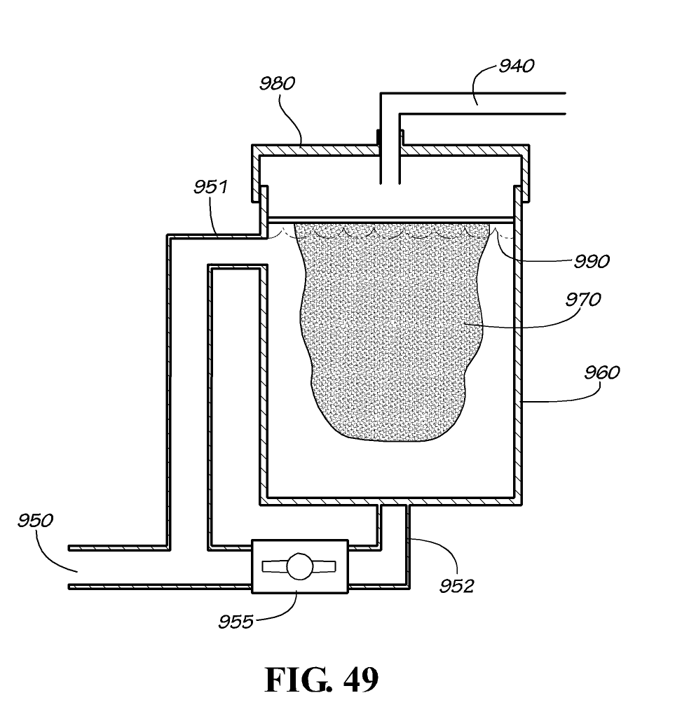

In another embodiment, a harvester unit has the capability to harvest microbes continuously and fixed time. FIG. 49 shows a harvester that has a rigid harvesting vessel 960 that houses a harvesting sieve 970 and is connected to the bioreactor harvesting outlet 910 via a connecting hose and solution inlet 940. This embodiment has two harvester outlets, one continuous harvesting outlet 951 and one fixed-time harvesting outlet 952. It also contains a valve 955 that when closed enables continuous harvesting, and when open, fixed-time harvesting. When the valve 955 is open, solution will flow through the fixed-time harvesting outlet 952 through the valve 955 to the bioreactor water inlet 920 returning the filtered solution back into the containing system 800 of bioreactor modules 050. Harvested microorganisms will collect in the sieve 970. The sieve 970 may be removed and the media harvested. When the valve 955 is closed, then the solution will flow through the continuous harvesting outlet 951 keeping the harvested media in suspension and alive until the time of harvest.

For continuous harvesting, before removing the sieve 970 containing harvested microorganisms, the pump delivering solution to the rigid harvesting vessel 960 should be disabled to stop the flow of solution and the valve 955 should be opened to allow water to drain from the harvested algae and harvesting unit. Upon removal of harvested media, a clean sieve 970 may be used to replace the sieve 970 laden with harvested microorganisms, and the system can be restarted by closing the valve 955 and enabling the solution delivery pump. In this way, harvested microorganisms may be removed from the site for processing at an alternate location.

Additional embodiments for harvesting include regulating solution flow into the filtration media in such a way that the quantity of harvested microorganism may be predetermined over a period of time. Embodiments may also include the use of nozzles on the solution inlet 940 that direct the flow of solution against the side of the sieve 970 enabling better filtration.

The present disclosure shows how harvesting can be applied to multiple, closed bioreactor modules 050 simultaneously (FIG. 45) for microorganisms using a simple cost-effective strainer method. The automation also speeds-up the process. Additionally, the harvesting station may function as a point of entry for additions to the system, such as nutrients, additional water, etc. They may all be added into the system through the harvester, without the need to open any of the individual bioreactor modules 050, which helps to prevent contamination.

The size of the system can vary. The optimum number of bioreactor modules 050 will be dependent on blower size, layout of the site, and some other parameters that influence how many bioreactor modules 050 make up each containing system 800 that is attached to each harvesting unit and how many containing systems 800 make up the overall bioreactor system.

In one embodiment, only a percentage of the microorganisms are harvested at a time leaving a remainder of microorganisms to repopulate the media. In one embodiment, 50% of the microorganisms are harvested each time. This leaves more than enough microorganisms in the filtrate to continue growth in the exponential phase until the next harvest. In another embodiment, 80% could be harvested and that would still leave enough microorganisms to repopulate the media. In another embodiment, even as much as 90% could be harvested and that would still leave enough microorganisms to repopulate the media; however, the duration until the next harvest would be longer. In one embodiment, the harvest frequency will keep the microorganism in its exponential growth stage.

There are many ways to determine if the system is ready for harvest. In one embodiment, one can determine the optical density of the water and microorganism to determine cell density. The cell density of each microorganism can be correlated with dry weight density, and so one can know when the microorganism is “ready” to be harvested. It is advantageous to harvest within a band that keeps the microbes in their exponential growth phase, so they are continuously growing as fast as possible. If the standing cell density in the bioreactors is either too low or too high, then they will not stay in the exponential growth phase and so the overall growth rate, and therefore microbial production, will be lower. If the system is not harvested regularly, or when “full,” then the microorganisms will continue to grow but much slower as it will fall out of the exponential growth phase. This is primarily due to factors such as the high microbial cell density reducing light penetration. Many microbes can stagnate for very long periods of time even for months, growing very slowly.

The present bioreactor system can be used to harvest microorganisms for many purposes, including biofuels, nutraceuticals, and food. Many bioreactor modules 050 cultivate microbes in open pond systems. These often have too many contaminates for these products. The design of the present disclosure is intended to function as a closed system with less risk of contamination and ease of harvesting.

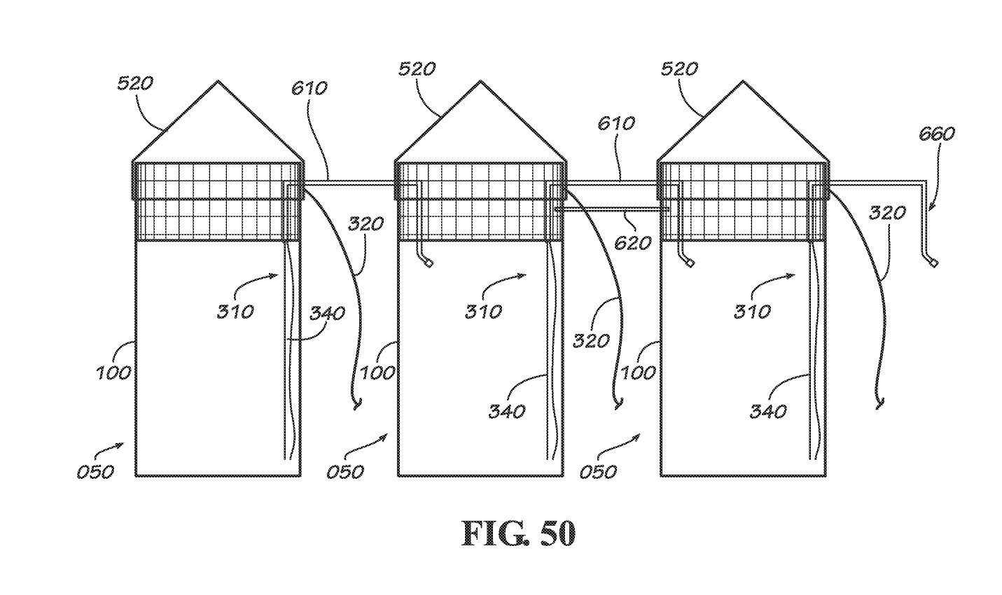

FIG. 50 shows three bioreactor modules 050 connected together. FIG. 50 shows the wire mesh support structure 100 and solution inside of the liner 250. Each bioreactor module 050 has its own gas delivery line 320, air lift circulation and aeration pump 310 and up-pipe 340, and cover 520. The three bioreactor modules 050 are connected by interconnecting tubes 610 and they have overflow conduit 620. The second and third bioreactor modules 050 have solution injector 660, though the solution interjector is depicted best outside, at the end of the system. For clarity, the support structure 100 has been cut away to better illustrate the interior components of the bioreactor module 050 from the outside.

Many different types of microbes may be used within the bioreactor module 050. In one embodiment, microbes that are effective at consuming carbon dioxide gas and growing rapidly are used. Other bioreactor modules 050, which use open pools regularly, rely on NaHCO₃ supplementation for nutrients. One of the advantages of the present system is that the bioreactor module 050 can rely on flue gas for its carbon source. Microbes that are better adapted to consuming carbon dioxide gas, as opposed to using NaHCO₃, will flourish better within the present bioreactor module 050.

In one embodiment, the temperature of the gas is 35 degrees Celsius. One way of lowering the temperature is by using a heat exchanger as depicted in FIG. 46. The heat exchanger may be either water or air cooled. In another embodiment, the gas temperature can be reduced by removing the heat from the flue gas in multiple stages, the harvested wet algae may be used to absorb some of that heat energy by using the piped hot gas, like a radiator, to heat a drying station. The flue gas would then be further cooled before entering into the gas delivery system.

FIG. 46 shows a schematic of one embodiment of a gas heat exchanger. Hot gas originates from a furnace stack 830 from any industrial source. The gas goes to a cooling and/or dehumidifying coil 840 and then through a blower 850. The gas leaving the blower 850 has been cooled and is ready to go to where it will be utilized. Water is needed for the coil 840 to operate. In one embodiment, water can be used from a river, through a river intake 892. A pump 880 may be used to create water flow. The water goes through a sand filter 870 and on to the cooling coil 840. The warmer water will leave the coil 840 out the condensate drain 860 and leave through the river water discharge 893.

In one embodiment, the cooling coil 840 has a cooling capacity suitable for flue gas on the air side. The coil 840 may be made of steel or coated copper. In one embodiment, the condensate will be low-pH and may need to be neutralized before discharging in the sewer or river.

In one embodiment, the blower 850 is a centrifugal utility vent that is sized for airflow and pressure drop through ductwork, cooling coil 840, and entry/exit losses, minus pressure available at furnace stack 830.

When the system includes many bioreactor modules 050, a large water sink may be used (all of the water in the system) to absorb some of the heat. Therefore, the flue gas entry temperature may be a little hotter; however, if it is too hot, then it will eventually just raise the temperature of the system too much and kill the microorganisms. Many microorganisms good for this system can survive in temperatures as high as 40+ deg C, but their optimal growth band is 32-38 deg C.

When industrial flue gas is accessible, it may be mixed with ambient air to a concentration level of 2-5% carbon dioxide to decrease the need for bicarbonate addition to the solution and to effectively scrub carbon dioxide emissions from adjacent facilities. When flue gas is not available, bottled carbon dioxide may be used. FIG. 46 shows that when flue gas is accessible, hot gas is run through a heat exchanger before being mixed with ambient air. Alternatively, flue gas may be mixed with ambient air before entering into heat exchanger in order to further sterilize ambient air with high temperature flue gas. In either case, cooled gas is fed through the primary air filter 730 and into the gas delivery backbone 810.

Flue gas temperature is often 180 degrees C. or higher, while outlet temperature should be 35 degrees C. or less. In some embodiments, the heat exchanger will have an outlet temp of 60-70 degrees C. and the hot gas will be utilized for drying wet algae, with an eventual target outlet temp from the dryer or 35 degrees C. or less.

In one embodiment, the microbes are prepared and acclimated for growth in the media within the bioreactor module 050. Microbes that have been naturally selected for thriving in a rich, gaseous carbon dioxide environment will grow faster, remove more carbon dioxide from the atmosphere, and create more microbial product, whether that be biofuels, nutraceuticals, or food product.

In one embodiment, Spirulina is used as the microbe within the bioreactor module 050. The Spirulina may be prepared before inoculation within the bioreactor module 050. This preparation procedure makes for a more flourishing strain of Spirulina that is good at feeding off gaseous carbon dioxide.

### Example 1

### Initial Inoculum to 18 L Carboys

1. Spirulina is grown on an agar slant for seven days. The Spirulina from the agar slant is then added to 50 mL of nutrient media in a 100 mL flask. In one embodiment, the nutrient media is Zarrouck's media (ZM). At least three flasks are prepared in this way. Each flask is prepared from a different agar slant.

2. These flasks are shaken using a laboratory shaker taker, or equivalent device, for 2 weeks at a speed of 100 rpm. If there are dregs after that, they will have to be filtered out.

3. After filtration, three of the flasks containing 50 mL of Spirulina mixture each are combined and mixed together so that there is 150 mL of Spirulina mixture.

4. This 150 mL of Spirulina mixture will be set on the shelf and given air for seven days to grow.

5. After that, the 150 mL of Spirulina mixture will be filtrated through gauze before using it to inoculate a larger flask or container. This is accomplished by adding the 150 mL of the mature Spirulina mixture to 650 mL of ZM in a 1000 mL flask.

6. Next, this 800 mL of Spirulina and ZM will be grown until it reaches an optical density (OD) at 560 nm wavelength of approximately 1.0 (as measured by a spectrophotometer). This is typically about seven days. This 800 mL of mature Spirulina mixture is now ready to be used as inoculum for larger flasks.

7. For 25% inoculum, 625 mL of the mature 800 mL Spirulina mixture is filtrated and added to 1875 mL of ZM in a 3000 mL flask. Or, for 30% inoculum, 750 mL of the mature 800 mL Spirulina mixture is filtrated and added to 1750 mL of ZM in a 3000 mL flask. Then the 25% or 30% inoculum in the 3000 mL flask is grown on the shelf for seven days. A 3000 mL flask which started with 25% inoculum should be used when it reaches an OD 560 nm of greater than or equal to 1.0. A 3000 mL flask that started with 30% inoculum should be used when the it reaches an OD 560 nm of less than 1.00—however, it should not be lower than 0.9. In each of these cases, the respective 3000 mL flask is considered to hold a mature Spirulina mixture which can then be used to inoculate other flasks or a larger container.

8. Steps 1-7 above should be repeated until several 3000 mL flasks of Spirulina mixture are mature. Then, for 30% inoculum, 5.4 L of the mature Spirulina mixture from 3000 mL flasks is added to 12.6 L of ZM in a 20 L carboy. After inoculation of the 20 L carboy, place the carboy outdoors in 7-8 hours per day of direct sunlight.

Step 8 is repeated until multiple carboys are prepared. Experiments were conducted where four such carboys were grown further with reduced amounts of NaHCO3 in their initial ZM composition, but with gaseous CO₂ injected as a replacement inorganic carbon source. One carboy had only 25% of the normal amount of NaHCO₃ in its ZM, another carboy had 50% of the normal amount of NaHCO₃ in its ZM, a third carboy had 75% of the normal amount of NaHCO₃ in its ZM, and the fourth carboy had the full normal amount of NaHCO₃ in its ZM. All four carboys had gaseous CO₂ injected. These examples show that the Spirulina grow better with gaseous CO₂ as a carbon source than NaHCO₃ alone, and that the amount of NaHCO₃ in the ZM for Spirulina can successfully be reduced through the addition of gaseous CO₂.

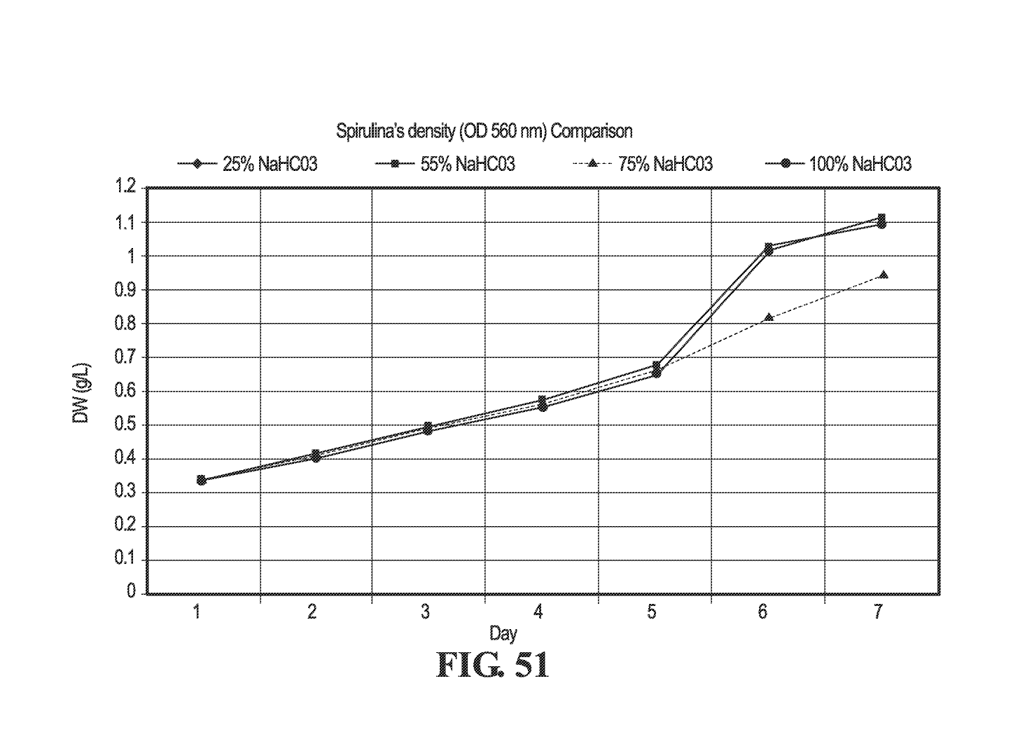

FIG. 51 shows the growth of the above mentioned carboys of Spirulina, and compares the optical density as each are grown using 25% NaHCO₃, 50% NaHCO₃, 75% NaHCO₃, and 100% NaHCO₃ in their ZM. The carboys were grown outdoors in sunlight.

### Example 2

### 25% NaHCO₃+CO₂

Table 1 shows the results for the carboy grown using 25% NaHCO₃ in its ZM and gaseous CO₂.

TABLE 1

| | 25% NaHCO₃ | | |
|---|---|---|---|
| Day | OD₅₆₀ | pH | DW (g/L) |
| 0 | 0.335 | 9.68 | 0.380 |
| 1 | 0.410 | 8.37 | 0.512 |
| Add CO₂ | | | |
| 2 | 0.492 | 8.60 | 0.584 |
| 3 | 0.575 | 9.35 | 0.588 |
| 4 | 0.665 | 9.75 | 0.648 |
| CO₂ tank runs | | | |
| out | | | |
| 7 | 1.025 | 10.18 | 0.856 |
| 8 | 1.098 | 10.32 | 1.148 |

### Example 3

### 50% NaHCO₃+CO₂

Table 2 shows the results for the carboy grown using 50% NaHCO₃ in its ZM and gaseous CO₂.

TABLE 2

| | 50% NaHCO₃ | | |
|---|---|---|---|
| Day | OD₅₆₀ | pH | DW (g/L) |
| 0 | 0.338 | 9.46 | 0.320 |
| 1 | 0.405 | 8.40 | 0.500 |
| Add CO₂ | | | |
| 2 | 0.490 | 8.61 | 0.564 |
| 3 | 0.572 | 9.31 | 0.580 |
| 4 | 0.670 | 9.62 | 0.648 |
| CO₂ tank runs | | | |
| out | | | |
| 7 | 1.027 | 10.07 | 0.928 |
| 8 | 1.115 | 10.16 | 1.192 |

### Example 4

### 75% NaHCO₃+CO₂

Table 3 shows the results for the carboy grown using 75% NaHCO₃ in its ZM and gaseous CO₂.

TABLE 3

| | 75% NaHCO₃ | | |
|---|---|---|---|
| Day | OD₅₆₀ | pH | DW (g/L) |
| 0 | 0.340 | 9.33 | 0.428 |
| 1 | 0.420 | 8.52 | 0.536 |
| Add CO₂ | | | |
| 2 | 0.495 | 8.68 | 0.580 |
| 3 | 0.572 | 9.35 | 0.592 |
| 4 | 0.667 | 9.63 | 0.664 |
| CO₂ tank runs | | | |
| out | | | |
| 7 | 0.815 | 9.85 | 0.812 |
| 8 | 0.945 | 9.91 | 0.936 |

### Example 5

### 100% NaHCO₃+CO₂

Table 4 shows the results for the carboy grown using 100% NaHCO₃ in its ZM and gaseous CO₂.

TABLE 4

| | 100% NaHCO₃ | | |
|---|---|---|---|
| Day | OD₅₆₀ | pH | DW (g/L) |
| 0 | 0.335 | 9.29 | 0.308 |
| 1 | 0.408 | 8.50 | 0.524 |
| 2 | 0.480 | 8.74 | 0.588 |
| Add CO₂ | | | |
| 3 | 0.553 | 9.44 | 0.596 |
| 4 | 0.652 | 9.73 | 0.640 |
| CO₂ tank runs | | | |
| out | | | |
| 7 | 1.015 | 10.17 | 1.020 |
| 8 | 1.110 | 10.25 | 1.244 |

### Results Comparison

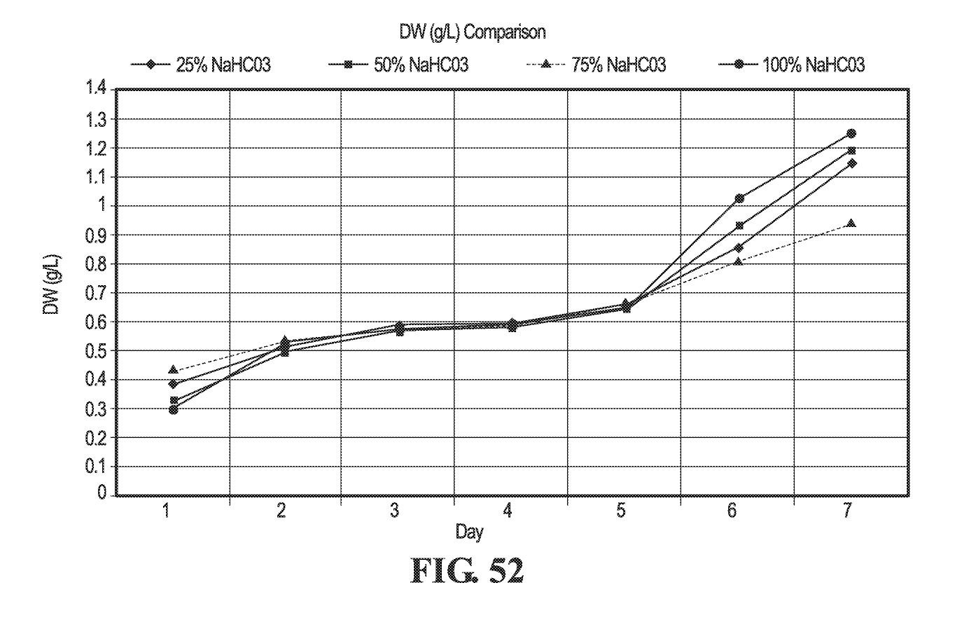

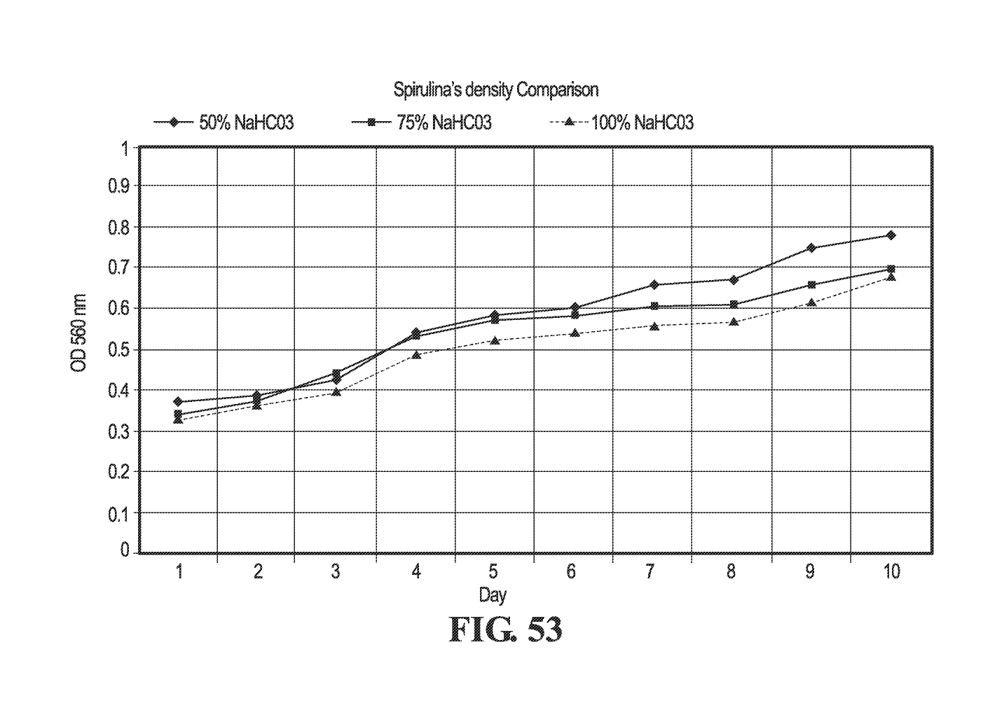

FIG. 52 shows the results for the four different carboys of Spirulina cultures, and compares the dry weight as grown in 25% NaHCO₃, 50% NaHCO₃, 75% NaHCO₃, and 100% NaHCO₃ in their respective ZM with gaseous CO₂ added.

Table 5 shows the densities of the Spirulina grown compared against the amount of NaHCO₃ in their respective ZM solutions that they were grown in.

TABLE 5

Spirulina's density (OD 560 nm)

| | 25% | 50% | 75% | 100% |
|---|---|---|---|---|
| Day | NaHCO₃ | NaHCO₃ | NaHCO₃ | NaHCO₃ |
| 0 | 0.335 | 0.338 | 0.340 | 0.335 |
| 1 | 0.410 | 0.405 | 0.420 | 0.408 |
| 2 | 0.492 | 0.490 | 0.495 | 0.480 |
| Add CO₂ | | | | |
| 3 | 0.575 | 0.572 | 0.572 | 0.553 |
| 4 | 0.665 | 0.670 | 0.667 | 0.652 |
| CO₂ tank | | | | |
| runs out | | | | |
| 7 | 1.025 | 1.027 | 0.815 | 1.015 |
| 8 | 1.098 | 1.115 | 0.945 | 1.110 |

Table 6 shows the dry weight of the Spirulina harvested compared against the amount of NaHCO₃ in their respective ZM solutions that they were grown in.

TABLE 6

DW (g/L)

| | 25% | 50% | 75% | 100% |
|---|---|---|---|---|
| Day | NaHCO₃ | NaHCO₃ | NaHCO₃ | NaHCO₃ |
| 0 | 0.380 | 0.320 | 0.428 | 0.308 |
| 1 | 0.512 | 0.500 | 0.536 | 0.524 |
| 2 | 0.584 | 0.564 | 0.580 | 0.588 |
| Add CO₂ | | | | |
| 3 | 0.588 | 0.580 | 0.592 | 0.596 |
| 4 | 0.648 | 0.648 | 0.664 | 0.640 |
| CO₂ tank | | | | |
| runs out | | | | |
| 7 | 0.856 | 0.928 | 0.812 | 1.020 |
| 8 | 1.148 | 1.192 | 0.936 | 1.244 |

In the above embodiments, the carboys of Spirulina mixture had gaseous CO₂ injected for four days. The pH of every carboy is reduced on the first day when gaseous CO₂ is added. After that, the pH slowly increased each day.

The Spirulina density (OD 560 nm) of every % NaHCO₃ carboy was quite similar except the 75% NaHCO₃ carboy which had OD, pH, and DW different than the other tanks on the seventh and eighth days.

The dry weight of every carboy was quite similar as well, and the highest DW was the 100% NaHCO₃ tank—but not by much.

Among these examples, the 50% NaHCO₃+CO₂ carboy showed the best results because the dry weight and optical density were similar to the 100% NaHCO₃ carboy, but 50% NaHCO₃ results in cost savings, and there's less precipitate.

It may be possible to grow algae in 25% NaHCO₃, but if it is grown for seven days using 50% NaHCO₃, it results in a higher yield of dry weight.

The next examples show results for carboys that were grown without CO₂ gas injection, and only the varying levels of % NaHCO₃ in their ZM.

FIG. 53 shows the results for three different carboys of Spirulina cultures, and compares the densities as grown in 50% NaHCO₃, 75% NaHCO₃, and 100% NaHCO₃ in their respective ZM.

### Example 6

### 50% NaHCO₃

Table 7 shows the results for the carboy grown using 50% NaHCO₃ in its ZM.

TABLE 7

| | 50% NaHCO₃ | | |
|---|---|---|---|
| Day | OD₅₆₀ | pH | DW (g/L) |
| 0 | 0.373 | 9.28 | 0.360 |
| 1 | 0.385 | 9.60 | 0.464 |
| 2 | 0.425 | 9.65 | 0.496 |
| 3 | 0.540 | 9.88 | 0.560 |
| 4 | 0.582 | 9.94 | 0.568 |
| 5 | 0.605 | 9.97 | 0.668 |
| 6 | 0.660 | 10.05 | 0.776 |
| 7 | 0.673 | 10.17 | 0.800 |
| 8 | 0.750 | 10.25 | 0.852 |
| 9 | 0.780 | 10.31 | 0.884 |

### Example 7

### 75% NaHCO₃

Table 8 shows the results for the carboy grown using 75% NaHCO₃ in its ZM.

TABLE 8

| | 75% NaHCO₃ | | |
|---|---|---|---|
| Day | OD₅₆₀ | pH | DW (g/L) |
| 0 | 0.340 | 9.20 | 0.392 |
| 1 | 0.372 | 9.55 | 0.460 |
| 2 | 0.440 | 9.60 | 0.552 |
| 3 | 0.530 | 9.89 | 0.576 |
| 4 | 0.572 | 9.95 | 0.576 |
| 5 | 0.585 | 9.98 | 0.592 |
| 6 | 0.603 | 10.03 | 0.680 |
| 7 | 0.610 | 10.15 | 0.716 |
| 8 | 0.660 | 10.25 | 0.760 |
| 9 | 0.698 | 10.29 | 0.820 |

### Example 8

### 100% NaHCO₃

Table 9 shows the results for the carboy grown using 100% NaHCO₃ in its ZM.

TABLE 9

| | 100% NaHCO₃ | | |
|---|---|---|---|
| Day | OD₅₆₀ | pH | DW (g/L) |
| 0 | 0.330 | 9.20 | 0.408 |
| 1 | 0.363 | 9.54 | 0.452 |
| 2 | 0.395 | 9.60 | 0.528 |
| 3 | 0.490 | 9.88 | 0.556 |
| 4 | 0.525 | 9.93 | 0.556 |
| 5 | 0.542 | 9.96 | 0.600 |
| 6 | 0.560 | 10.06 | 0.720 |
| 7 | 0.570 | 10.13 | 0.756 |
| 8 | 0.618 | 10.22 | 0.784 |
| 9 | 0.680 | 10.24 | 0.880 |

### Results Comparison

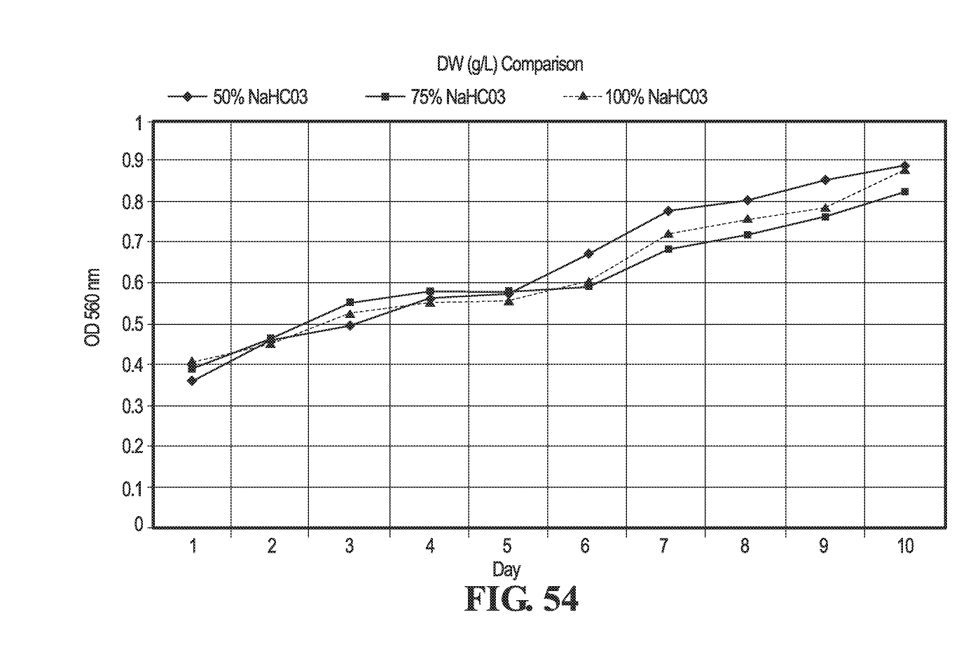

FIG. 54 shows the results for the three different carboys of Spirulina cultures, and compares the densities as grown in 50% NaHCO₃, 75% NaHCO₃, and 100% NaHCO₃ in their respective ZM.

Table 10 shows the densities of the Spirulina compared against the % NaHCO₃

TABLE 10

| | Spirulina's Density (OD 560 nm) | | |
|---|---|---|---|
| Day | 50% NaHCO₃ | 75% NaHCO₃ | 100% NaHCO₃ |
| 0 | 0.373 | 0.340 | 0.330 |
| 1 | 0.385 | 0.372 | 0.363 |
| 2 | 0.425 | 0.440 | 0.395 |
| 3 | 0.540 | 0.530 | 0.490 |
| 4 | 0.582 | 0.572 | 0.525 |
| 5 | 0.605 | 0.585 | 0.542 |
| 6 | 0.660 | 0.603 | 0.560 |
| 7 | 0.673 | 0.610 | 0.570 |
| 8 | 0.750 | 0.660 | 0.618 |
| 9 | 0.780 | 0.698 | 0.680 |

Table 11 shows the dry weights of the Spirulina compared against the % NaHCO₃

TABLE 11

| | DW (g/L) | | |
|---|---|---|---|
| Day | 50% NaHCO₃ | 75% NaHCO₃ | 100% NaHCO₃ |
| 0 | 0.360 | 0.392 | 0.408 |
| 1 | 0.464 | 0.460 | 0.452 |
| 2 | 0.496 | 0.552 | 0.528 |
| 3 | 0.560 | 0.576 | 0.556 |
| 4 | 0.568 | 0.576 | 0.556 |
| 5 | 0.668 | 0.592 | 0.600 |
| 6 | 0.776 | 0.680 | 0.720 |
| 7 | 0.800 | 0.716 | 0.756 |
| 8 | 0.852 | 0.760 | 0.784 |
| 9 | 0.884 | 0.820 | 0.880 |

Spirulina that had been cultured within 50% NaHCO₃ have Spirulina density (OD value) higher than 75% and 100% NaHCO₃

The dry weight of Spirulina grow with 50% NaHCO₃ in its ZM is the highest.

It should be emphasized that the embodiments described herein are merely possible examples of implementations, merely set forth for a clear understanding of the principles of the present disclosure. Many variations and modifications may be made to the described embodiment(s) without departing substantially from the spirit and principles of the present disclosure. Further, the scope of the present disclosure is intended to cover any and all combinations and sub-combinations of all elements, features, and aspects discussed above. All such modifications and variations are intended to be included herein within the scope of the present disclosure, and all possible claims to individual aspects or combinations of elements or steps are intended to be supported by the present disclosure.

One should note that conditional language, such as, among others, “can,” “could,” “might,” or “may,” unless specifically stated otherwise, or otherwise understood within the context as used, is generally intended to convey that certain embodiments include, while alternative embodiments do not include, certain features, elements and/or steps. Thus, such conditional language is not generally intended to imply that features, elements and/or steps are in any way required for one or more particular embodiments or that one or more particular embodiments necessarily include logic for deciding, with or without user input or prompting, whether these features, elements and/or steps are included or are to be performed in any particular embodiment. Unless stated otherwise, it should not be assumed that multiple features, embodiments, solutions, or elements address the same or related problems or needs.

Various implementations described in the present disclosure may include additional systems, methods, features, and advantages, which may not necessarily be expressly disclosed herein but will be apparent to one of ordinary skill in the art upon examination of the following detailed description and accompanying drawings. It is intended that all such systems, methods, features, and advantages be included within the present disclosure and protected by the accompanying claims.

1. A bioreactor comprising:
a support structure, and
a liner, wherein the liner is fitted within the support structure.

2. The bioreactor of claim 1, wherein the liner is translucent.

3. The bioreactor of claim 1, further comprising:
a gas delivery line, wherein the gas delivery line enters the bioreactor through a puncture in the liner.

4. The bioreactor of claim 1, further comprising:
an aeration pump, wherein the aeration pump includes an up-pipe and a gas delivery line connected to the up-pipe.

5. The bioreactor of claim 4, wherein the aeration pump further comprises:
an airlift geyser, wherein a second gas delivery line is used to deliver gas into a secondary gas chamber at the bottom of the aeration pump.

6. The bioreactor of claim 1, further comprising:
a cover substantially covering the top of the support structure.

7. The bioreactor of claim 1, further comprising:
interconnecting tubes, wherein the interconnecting tubes connect the bioreactor to at least one other bioreactor.

8. The bioreactor of claim 1, further comprising:
a solution injector port, wherein the solution injector port is connected to interconnecting tubes on the inside of the bioreactor.

9. The bioreactor of claim 1, wherein the bioreactor is connected to at least one other bioreactor and wherein at least one bioreactor is connected to a harvesting station.

10. The bioreactor of claim 1, wherein the bioreactor is connected to a harvesting station.

11. The bioreactor of claim 10, wherein the harvesting station comprises:
a rigid harvesting vessel, a harvesting sieve, a solution inlet, and a harvesting outlet,
wherein the harvesting sieve is positioned inside the rigid harvesting vessel,
wherein the rigid harvesting vessel has a harvesting outlet, and
wherein the harvesting outlet may be opened or closed.

12. The bioreactor of claim 10, wherein the harvesting station comprises:
a rigid harvesting vessel, a harvesting sieve, a solution inlet, an upper harvesting outlet and a lower harvesting outlet, and a valve,
wherein the harvesting sieve is positioned in the rigid harvesting vessel to capture or receive the solution or media that enters the harvesting station from the solution inlet,
wherein the upper harvesting outlet is located on the upper half of the rigid harvesting vessel and the lower harvesting outlet is located on the lower half of the rigid harvesting vessel,
wherein the valve may be opened and the solution exits the lower harvesting outlet or closed allowing the solution to leave the lower harvesting outlet, and
wherein the harvesting outlet may be opened to allow the solution to exit from the harvesting station.

13. A containing system of bioreactors, the containing system comprising:
a plurality of individual interconnected bioreactors that are functionally self-supporting for the growth of microbes, wherein each bioreactor is comprised of a fixed container holding a column of a solution, wherein the solution comprises water, nutrients, and microbes, and
interconnecting tubes that allow the solution to move from one bioreactor to another bioreactor, and wherein the bioreactors are interconnected in series forming a closed loop.

14. The containing system of claim 13, further comprising:
a gas delivery backbone that is connected to and introduces gas into the gas delivery lines.

15. The containing system of claim 14, further comprising:
a blower, wherein the blower is connected to the gas delivery backbone.

16. The containing system of claim 15, further comprising:
an air filter, wherein the air filter is connected between the blower and the gas delivery backbone.

17. The containing system of claim 16, further comprising:
a microbial filter, wherein the microbial filter is connected between the air filter and the gas delivery backbone.

18. The containing system of claim 13, further comprising:
a harvesting module that is connected in series with the other bioreactors in the containing system and within the closed loop,
wherein the harvesting module may be connected at any interconnection point within the containing system through interconnection tubes, and
wherein the harvesting module has an interconnecting tube that receives solution from one bioreactor in the containing system and another interconnecting tube that expels solution to the next bioreactor in series.

19. A method of reducing carbon dioxide emissions, the method comprising:
introducing gaseous carbon dioxide to a bioreactor
wherein the bioreactor includes a support structure and a transparent liner; and wherein the bioreactor includes media containing microbes that consume gaseous carbon dioxide as a carbon source, and
growing microbes in the bioreactor.

20. A method of claim 19, the method further comprising:
inoculating the bioreactor with microbes that are acclimated to a low amount of NaHCO3 and gaseous carbon dioxide.

21. A method of claim 20, wherein the amount of NaHCO3 is less than 14.0 g NaHCO3 per L.

22. A method of harvesting microorganisms from a containing system, the method comprising:
emptying the solution from the solution outlet of a harvesting unit of a containing system,
wherein the containing system comprises a plurality of bioreactors wherein each bioreactor is comprised of a support structure and a transparent liner,
wherein the harvesting unit is closed off from all the other bioreactors in the containing system, and
removing a sieve from the harvesting unit,
wherein the sieve contains microbes.

23. A method of claim 22, the method further comprising:
adding nutrients and water to the bioreactor.

24. The containing system of claim 13, each bioreactor within the containing system further comprising:
an aeration or circulation pump that includes an up-pipe and a gas delivery line,
wherein the aeration or circulation pump is inside the bioreactor and extends to the bottom of the bioreactor,
wherein the opening of the gas delivery line is inside of the bottom of the up-pipe.

25. The containing system of claim 24, each bioreactor within the containing system further comprising:
a second gas delivery line, and
a secondary chamber at the bottom of the aeration pump, wherein the second gas delivery line opens into the secondary chamber.

26. The containing system of claim 24, each bioreactor within the containing system further comprising:
an interconnecting tube that delivers solution from the up-pipe of one bioreactor to the solution of a connecting bioreactor.

27. The containing system of claim 24, each bioreactor within the containing system further comprising:
a solution injector tube, wherein the solution injector tube is connected to the interconnecting tubes of the bioreactor, and wherein the solution injector tube has an opening to the inside of the bioreactor.

28. The containing system of claim 27, wherein the solution injector tube is at an angle of about 10 to 45 degrees.
